(19) United States
(12) Patent Application Publication
(10) Pub. No.: US 2026/0285833 A1
(43) Pub. Date: Sep. 24, 2026

(54) SYNTHESIS OF MAVORIXAFOR AND INTERMEDIATES THEREOF

(71) Applicant: X4 Pharmaceuticals, Inc., Boston, MA (US)

(72) Inventors: Angelina Roberta Sekirnik, Logatec (SI); Art Taveras, Boston, MA (US); Roger Hanselmann, Boston, MA (US); Feng Gao, Shanghai (CN); Zhigang Zhao, Shanghai (CN); Tao Xiao, Chengdu (CN)

(21) Appl. No.: 19/474,600

(22) PCT Filed: Apr. 12, 2024
(86) PCT No.: PCT/US2024/024356

§ 371 Date: Oct. 10, 2025

(30) Foreign Application Priority Data
Apr. 13, 2023 (WO) PCT/CN2023/088078

(51) Int. Cl. C07D 401/12; C07D 215/40

(52) U.S. Cl. CPC C07D 401/12; C07D 215/40

(57) ABSTRACT

The present invention relates to methods for synthesizing the C-X-C receptor type 4 (CXCR4) inhibitor mavorixafor and to intermediates thereto.

### CROSS-REFERENCE TO RELATED APPLICATIONS

This application claims priority to International Patent Application Number PCT/CN2023/088078, filed Apr. 13, 2023, the contents of which are incorporated herein by reference.

### FIELD OF THE INVENTION

The present invention relates to methods for synthesizing mavorixafor, and to intermediates thereto.

### BACKGROUND OF THE INVENTION

C-X-C chemokine receptor type 4 (CXCR4), also known as fusin or cluster of differentiation 184 (CD 184), is a seven transmembrane G-protein coupled receptor (GPCR) belonging to Class I GPCR or rhodopsin-like GPCR family. Under normal physiological conditions, CXCR4 carries out multiple roles and is principally expressed in the hematopoietic and immune systems. CXCR4 was initially discovered as one of the co-receptors involved in human immunodeficiency virus (HIV) cell entry. Subsequent studies showed that it is expressed in many tissues, including brain, thymus, lymphatic tissues, spleen, stomach, and small intestine, and also specific cell types such as hematopoietic stem cells (HSC), mature lymphocytes, and fibroblasts. CXCL12, previously designated SDF-la, is the only known ligand for CXCR4. CXCR4 mediates migration of stem cells during embryonic development as well as in response to injury and inflammation. Multiple roles have been demonstrated for CXCR4 in human diseases such as cellular proliferative disorders, Alzheimer's disease, HIV, rheumatoid arthritis, pulmonary fibrosis, and others. For example, expression of CXCR4 and CXCL12 have been noted in several tumor types. CXCL12 is expressed by cancer-associated fibroblast (CAFs) and is often present at high levels in the tumor microenvironment (TME). In clinical studies of a wide range of tumor types, including breast, ovarian, renal, lung, and melanoma, expression of CXCR4/CXCL12 has been associated with a poor prognosis and with an increased risk of metastasis to lymph nodes, lung, liver, and brain, which are sites of CXCL12 expression. CXCR4 is frequently expressed on melanoma cells, particularly the CD133+ population that is considered to represent melanoma stem cells; in vitro experiments and murine models have demonstrated that CXCL12 is chemotactic for such cells.

Peripheral leukocyte deficiency is a common feature of multiple diseases and may render affected individuals susceptible to infections, both common and opportunistic. The CXCR4 chemokine receptor regulates the trafficking of leukocytes among the bone marrow, blood, and lymphatic system (Al Ustwani O, et al. Br J Haematol. 2014; 164:15-23). It is known to be involved with some immunodeficiencies such as WHIM syndrome, but not all human primary immunodeficiency diseases (PIDs) involve a mutation or alteration in CXCR4 function. PIDs comprise 330 distinct disorders with 320 different gene defects listed. Long considered as rare diseases, recent studies tend to show that they are more common than generally thought, if only by their rapidly increasing number. The International Union of Immunological Societies (IUIS) PID expert committee proposed a PID classification since 1999, which facilitates clinical research and comparative studies worldwide; it is updated every other year to include new disorders or disease-causing genes.

For example, neutropenia is a condition characterized by an abnormally low concentration of neutrophils circulating in the blood, and defined by an absolute neutrophil count (ANC) below 1500 cells/μL. Severe neutropenia (ANC <500 cells/μL) is a risk factor for susceptibility to bacterial infection. Neutrophils make up the majority of circulating white blood cells and play an important role in the body's defenses against bacterial or fungal pathogenic infections and in shaping the host response to infection. In addition, neutrophils participate in immune system homeostasis. Neutropenia can be divided into congenital (i.e., present at birth) and acquired. Additionally, neutropenia can be “acute” (transient, or temporary, often as a response to specific events that deplete the body of neutrophils, such as radiation or chemotherapy), or “chronic” (a long-term or long-lasting effect that may be due to the presence of genetic abnormalities).

Acute or transient neutropenia can be caused by infectious agents, such as the typhoid-causing bacterium Salmonella enterica; and cytomegalovirus, as well as chemical agents, including propylthiouracil; levamisole; penicillamine; clozapine; valproic acid; and cancer chemotherapy. Chronic neutropenia can be caused by genetic abnormalities (congenital neutropenia). Mutations in ELANE are the most common cause of congenital neutropenia. Other examples of genes that can be responsible for genetic causes of neutropenia include HAX1, G6PC3, WAS, SBDS, and others. In addition, some enzyme deficiencies can be associated with neutropenia such as glycogen storage disease 1b. Other causes of neutropenia include mitochondrial diseases, such as Pearson syndrome. Some autoimmune diseases, such as systemic lupus erythematosus (“SLE” or “lupus”) may be associated with neutropenia. Aplastic anemia, due to bone marrow failure, is associated with thrombocytopenia, anemia and neutropenia; Evans syndrome is characterized by autoimmune hemolytic anemia (AIHA) and immune thrombocytopenia (ITP) and/or immune neutropenia; and Felty's syndrome is characterized by rheumatoid arthritis, splenomegaly and neutropenia. Chronic neutropenia may also be the result of nutritional deficiencies, such as abnormally low levels of copper or Vitamin B12; or chronic infections, such as with human immunodeficiency virus (HIV), the agent that causes AIDS.

Neutropenia may be asymptomatic and often is only diagnosed fortuitously. Today, the standard treatment for severe neutropenia is administration of granulocyte colony-stimulating factor (G-CSF). Historically, neutropenia has been treated in a host of manners, including splenectomy, corticosteroids, androgens, and immunosuppressive and immune-modulating therapies. Currently, however, these treatments are generally not recommended except in cases where treatment with G-CSF is not effective. Dale et al. (2017) Curr. Opin. Hematol. 24:46-53; Sicre de Fontbrune et al. (2015) Blood 126:1643-1650. Other treatments for neutropenia can include bone marrow transportation and/or treatment with cord blood stem cells.

There exists a need for improved treatments capable of reversing peripheral leukocyte deficiencies, neutropenia, and other immune cell deficiencies. The present invention addresses this need and provides other related advantages. It has now been found that CXCR4 inhibitors such as mavorixafor (X4P-001) are useful for treating diseases and disorders related to immune-deficiencies, or diseases and disorders which present as imbalances in levels of one or more immune cells in a subject.

Furthermore, there is evidence implicating the CXCL12/CXCR4 axis in contributing to the loss or lack of tumor responsiveness to angiogenesis inhibitors (also referred to as “angiogenic escape”). In animal cancer models, interference with CXCR4 function has been demonstrated to alter the TME and sensitize the tumor to immune attack by multiple mechanisms such as elimination of tumor re-vascularization and increasing the ratio of CD8+ T cells to Treg cells. These effects result in significantly decreased tumor burden and increased overall survival in xenograft, syngeneic, and transgenic cancer models. See Vanharanta et al. (2013) Nat Med 19:50-56; Gale and McColl (1999) BioEssays 21:17-28; Highfill et al. (2014) Sci Transl Med 6: ra67; Facciabene et al. (2011) Nature 475:226-230.

The above underscores the significant unmet need for CXCR4 inhibitors to treat the many diseases and conditions mediated by aberrant or undesired expression of the CXCR4 receptor, and to correct imbalances of cells of the immune system categorized as lymphocytes (T-cells, B-cells and NK cells), neutrophils, and monocytes/macrophages, for example in primary immunodeficiency disorders, neutropenia and cellular proliferative disorders.

### DETAILED DESCRIPTION OF THE INVENTION

Mavorixafor, and pharmaceutically acceptable compositions thereof, are effective as CXC receptor type 4 (CXCR4) inhibitors, and are useful for treating a variety of diseases, disorders, or conditions associated with CXCR4, such as hyperproliferative conditions including various cancers. In some embodiments, mavorixafor or a pharmaceutically acceptable salt thereof is prepared using a method disclosed herein. The methods and intermediates of the present invention are useful for preparing mavorixafor, which is described in, e.g., U.S. patent application Ser. No. 16/215,963 (U.S. Pat. No. 10,548,889), in the name of Brands, the entirety of which is incorporated herein by reference. The present invention relates to certain novel intermediates to the synthesis of mavorixafor that are easier to isolate, purify, and/or handle, and/or are more stable in comparison to corresponding intermediates in the existing methods. Furthermore, such intermediates enable methods of synthesis of mavorixafor that feature improved efficiency, reproducibility, purity, scalability, and manufacturability. In certain embodiments, the present compounds are generally prepared through intermediates A and B, according to Scheme I set forth below:

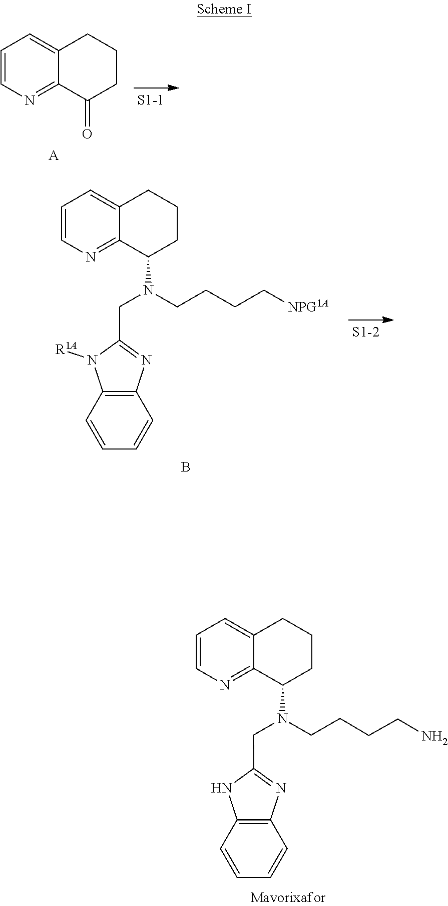

In Scheme I above, R^{1A} is H, or a suitable amino protecting group; and PG^{1A} is any combination of two amino protecting groups, a single amino protecting group and H, or two H. PG^{1A} may also be, taken with the nitrogen to which it is bound, a cyclic imide. Additionally, PG^{1A} may also be absent and a triple bond present between the corresponding nitrogen and the carbon to which the nitrogen is bound, such that the nitrogen and the carbon to which it is bound form a nitrile. In step S1-1, a ketone of the formula A is converted to a compound of formula B. In step S1-2, the compound of formula B is unmasked to prepare mavorixafor.

### Preparation of Mavorixafor

In one aspect, the present invention provides a method of preparing mavorixafor, or a pharmaceutically acceptable salt thereof, as set forth in Scheme II below.

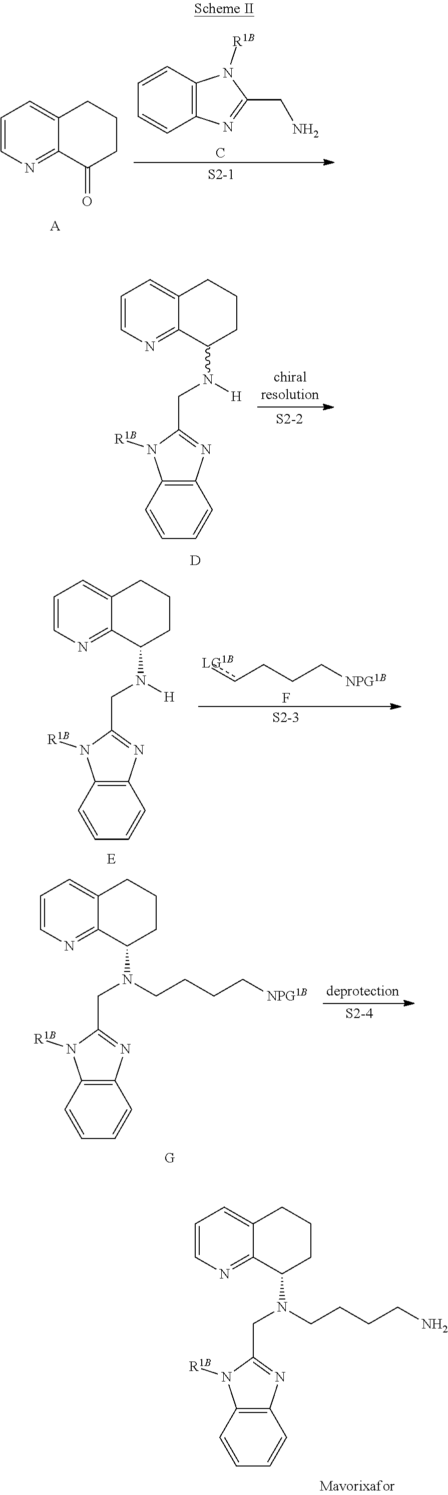

In one aspect, the present invention provides methods for preparing compounds of formulae C, D, E, F, G, and salts thereof, and mavorixafor and pharmaceutically acceptable salts thereof, according to the steps depicted in Scheme II. In the compounds of the present formulae, R^{1B} is H, or a suitable benzimidazole protecting group; LG^{1B} is ═O, a suitable leaving group, or, taken with the carbon atom to which it is bound, an acetal; and PG^{1B} is any combination of two amino protecting groups, a single amino protecting group and H, or two H. PG^{1B} may also be, taken with the nitrogen to which it is bound, a cyclic imide. Additionally, PG^{1B} may be absent and a triple bond present between the corresponding nitrogen and the carbon to which the nitrogen is bound, such that the nitrogen and the carbon to which it is bound form a nitrile. It is understood in the art that a nitrile can function as a masked amino group. Thus, deprotection is understood to include reduction of a nitrile to an amino group.

Suitable amino protecting groups are well known in the art and include those described in detail in Protecting Groups in Organic Synthesis, T. W. Greene and P. G. M. Wuts, 3rd edition, John Wiley & Sons, 1999, the entirety of which is incorporated herein by reference.

Cyclic imides are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. In some embodiments, PG^{1B} succinimide, maleimide, or phthalimide.

Acetals are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. In some embodiments, LG^{1B}, taken with the carbon to which it is bound, is 1,3-dioxane or 1,3-dioxolane.

Suitable benzimidazole protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. In certain embodiments, R^{1B}, taken with the nitrogen atom to which it is bound, is selected from, but are not limited to, aralkylamines, carbamates, allyl amines, amides, and the like, e.g., t-butyloxycarbonyl (Boc), ethyloxycarbonyl, methyloxycarbonyl, trichloroethyloxycarbonyl, allyloxycarbonyl (Alloc), benzyloxycarbonyl (CBZ), allyl, benzyl (Bn), fluorenylmethylcarbonyl (Fmoc), acetyl, chloroacetyl, dichloroacetyl, trichloroacetyl, phenylacetyl, trifluoroacetyl, benzoyl, pivaloyl and the like.

Leaving groups are well known in the art and include those described in detail in Leaving group, Gold Book, IUPAC. 2009, ISBN 978-0-9678550-9-7, the entirety of which is incorporated herein by reference.

When [symbol] is used to join two atoms, it denotes a covalent double bond between the two atoms that may be of any geometry (cis or trans).

In step S2-1, a compound of formula C is coupled with a compound of formula A to prepare a compound of formula D. In some embodiments, the compound of formula C and the compound of formula A are coupled through reductive amination. Methods for reductive amination are well known in the art. In some embodiments, R^{1B} is H.

In step S2-2, the compound of formula D is purified to prepare the enantiomerically enriched compound E. A compound is said to be enantiomerically enriched if the enantiomeric excess exceeds 70%. In some embodiments, the enantiomeric excess is greater than 80%. In some embodiments, the enantiomeric excess is greater than 90%. In some embodiments, the enantiomeric excess is greater than 95%. In some embodiments, the enantiomeric excess is greater than 97%. In some embodiments, the enantiomeric excess is greater than 99%. In some embodiments, the enantiomeric excess is greater than 99.9%.

In some embodiments, the compound of formula D is purified to prepare the enantiomerically enriched compound of formula E through recrystallization using a chiral acid. Chiral acids are well known in the art and include, but are not limited to, (S)-2-(6-methoxynaphthalen-2-yl) propanoic acid, L-(+)-tartaric acid, (−)-di-p-toluoyl-L-tartaric acid, 2,3-di-O-para-toluoyl-D-tartaric acid, (−)-di-p-toluoyl-L-tartaric acid, (−)-dibenzoyl-L-tartaric acid, (+)-di-p-anisoyl-D-tartaric acid, L-malic acid, (S)-(+)-mandelic acid, (+)-camphoric acid, L-pyroglutamic acid, (S)-2-(6-methoxynaphthalen-2-yl) propanoic acid, N-Boc-D-proline, N-Boc-L-alanine, N-acetyl-L-tyrosine, N-acetyl-L-leucine, N-acetyl-L-proline, N-tosyl-L-proline, (1S)-(+)-10-camphorsulfonicacid, (1S)-(+)-10-camphorsulfonic acid, (1R)-(−)-10-camphorsulfonic acid, (S)-BNP acid, (S)-(+)-BNP, or (S)-Naproxen.

In some embodiments, the chiral acid is selected from one or more of (S)-2-(6-methoxynaphthalen-2-yl) propanoic acid, L-(+)-tartaric acid, (−)-di-p-toluoyl-L-tartaric acid, 2,3-di-O-para-toluoyl-D-tartaric acid, (−)-di-p-toluoyl-L-tartaric acid, (−)-dibenzoyl-L-tartaric acid, (+)-di-p-anisoyl-D-tartaric acid, (S)-(+)-mandelic acid, (+)-camphoric acid, L-pyroglutamic acid, (S)-2-(6-methoxynaphthalen-2-yl) propanoic acid, N-Boc-L-alanine, N-acetyl-L-tyrosine, N-acetyl-L-leucine, (1S)-(+)-10-camphorsulfonicacid, (1R)-(−)-10-camphorsulfonic acid, or (S)-(+)-BNP.

In some embodiments, the compound of formula D is purified to prepare the enantiomerically enriched compound of formula E through crystallization using one or more solvents selected from MeCN, THF, DCM, iPrOH, EA, EtOH or acetone.

In some embodiments, up to 0.5 equivalents of chiral acid is used relative to the compound of formula D. In some embodiments, between 0.5 and 1.5 equivalents of chiral acid is used relative to the compound of formula D. In some embodiments, between 1.0 and 2.5 equivalents of chiral acid is used relative to the compound of formula D.

In some embodiments, the compound of formula D is dissolved in solvent with the chiral acid at an elevated temperature before it is cooled down. In some embodiments, the compound of formula D is dissolved in solvent with the chiral acid at room temperature before it is heated to an elevated temperature, following which the mixture is cooled down. In some embodiments, the product collected from one crystallization is used to perform additional crystallizations.

In some embodiments, the compound of formula D is initially dissolved in solvent at a concentration of up to 0.25 g/mL.

In some embodiments, the compound of formula D is recrystallized using up to 2.0 equivalents of (R)-(−)-mandelic acid, and MeCN as a solvent. In some embodiments, the compound of formula D is dissolved in MeCN at a concentration of up 0.25 g/mL, to which up to 2.0 equivalents of (R)-(−)-mandelic acid is added. In some embodiments, the compound of formula D is dissolved in MeCN at a concentration of up 0.25 g/mL, to which up to 2.0 equivalents of (R)-(−)-mandelic acid is added at room temperature, at which point it is heated to an elevated temperature and held at that temperature for a period of time before it is cooled down and held at that temperature for a period of time. In some embodiments, the compound of formula D is dissolved in MeCN at a concentration of up 0.25 g/mL, to which up to 2.0 equivalents of (R)-(−)-mandelic acid is added at 15-25° C., at which point it is heated to a temperature between 65-85° C. and held at that temperature for 0.1 to 3 hours before it is cooled down to 0-25° C. and held at that temperature for 1 to 10 hours. In some embodiments, the compound of formula D is dissolved in MeCN at a concentration of up 0.25 g/mL, to which up to 2.0 equivalents of (R)-(−)-mandelic acid is added at 15-25° C., at which point it is heated to a temperature between 65-85° C. and held at that temperature for 0.1 to 3 hours before it is cooled down to 0-25° C. and held at that temperature for 1 to 10 hours, at which point the crystallized product is collected and used for additional rounds of recrystallizations using similar conditions, which yields the compound of formula E with an enantiomeric excess of at least 90%.

In some embodiments, the compound of formula D is dissolved in MeCN at a concentration of up 0.25 g/mL, to which up to 2.0 equivalents of (R)-(−)-mandelic acid is added at 15-25° C., at which point it is heated to a temperature between 65-85° C. and held at that temperature for 0.1 to 3 hours before it is cooled down to 0-25° C. and held at that temperature for 1 to 10 hours, at which point the crystallized product is collected and used for additional rounds of recrystallizations using similar conditions, which yields the compound of formula E with an enantiomeric excess of at least 95%.

In some embodiments, the compound of formula D is dissolved in MeCN at a concentration of up 0.25 g/mL, to which up to 2.0 equivalents of (R)-(−)-mandelic acid is added at 15-25° C., at which point it is heated to a temperature between 65-85° C. and held at that temperature for 0.1 to 3 hours before it is cooled down to 0-25° C. and held at that temperature for 1 to 10 hours, at which point the crystallized product is collected and used for additional rounds of recrystallizations using similar conditions, which yields the compound of formula E with an enantiomeric excess of at least 99%.

In some embodiments, the compound of formula D is recrystallized using 1.0 equivalents of (R)-(−)-mandelic acid, and MeCN as a solvent. In some embodiments, the compound of formula D is dissolved in MeCN at a concentration of up 0.11 g/mL, to which 1.0 equivalents of (R)-(−)-mandelic acid is added. In some embodiments, the compound of formula D is dissolved in MeCN at a concentration of up 0.11 g/mL, to which 1.0 equivalents of (R)-(−)-mandelic acid is added at room temperature, at which point it is heated to an elevated temperature and held at that temperature for a period of time before it is cooled down and held at that temperature for a period of time. In some embodiments, the compound of formula D is dissolved in MeCN at a concentration of up 0.11 g/mL, to which 1.0 equivalents of (R)-(−)-mandelic acid is added at 20-25° C., at which point it is heated to a temperature between 80-85° C. and held at that temperature for 0.5 hours before it is cooled down to 20-25° C. and held at that temperature for 3 hours. In some embodiments, the compound of formula D is dissolved in MeCN at a concentration of up 0.11 g/mL, to which 1.0 equivalents of (R)-(−)-mandelic acid is added at 20-25° C., at which point it is heated to a temperature between 80-85° C. and held at that temperature for 0.5 hours before it is cooled down to 20-25° C. and held at that temperature for 3 hours, at which point the crystallized product is collected and used for additional rounds of recrystallizations using similar conditions, which yields the compound of formula E with an enantiomeric excess of at least 90%.

In some embodiments, the compound of formula D is dissolved in MeCN at a concentration of up 0.11 g/mL, to which 1.0 equivalents of (R)-(−)-mandelic acid is added at 20-25° C., at which point it is heated to a temperature between 80-85° C. and held at that temperature for 0.5 hours before it is cooled down to 20-25° C. and held at that temperature for 3 hours, at which point the crystallized product is collected and used for additional rounds of recrystallizations using similar conditions, which yields the compound of formula E with an enantiomeric excess of at least 95%.

In some embodiments, the compound of formula D is dissolved in MeCN at a concentration of up 0.11 g/mL, to which 1.0 equivalents of (R)-(−)-mandelic acid is added at 20-25° C., at which point it is heated to a temperature between 80-85° C. and held at that temperature for 0.5 hours before it is cooled down to 20-25° C. and held at that temperature for 3 hours, at which point the crystallized product is collected and used for additional rounds of recrystallizations using similar conditions, which yields the compound of formula E with an enantiomeric excess of at least 99%.

In some embodiments, the compound of formula D is recrystallized using up to 1.5 equivalents of (1R)-(−)-10-camphorsulfonic acid, and THE as a solvent. In some embodiments, the compound of formula D is dissolved in THF at a concentration of up to 0.2 g/mL, to which up to 1.5 equivalents of (1R)-(−)-10-camphorsulfonic acid is added. In some embodiments, the compound of formula D is dissolved in THF at a concentration of up to 0.2 g/mL, to which up to 1.5 equivalents of (1R)-(−)-10-camphorsulfonic acid is added at room temperature and held at that temperature for a period of time, at which point it is heated to an elevated temperature and held at that temperature for a period of time, following which it is cooled and held at that temperature for a period of time. In some embodiments, the compound of formula D is dissolved in THF at a concentration of up to 0.2 g/mL, to which up to 1.5 equivalents of (1R)-(−)-10-camphorsulfonic acid is added at a temperature between 10-30° C. and held at that temperature for up to 3 hours before it is heated up to 50-75° C. and held at that temperature for up to 3 hours, following which the mixture is cooled to a temperature between 0-25° C. and held at that temperature for up to 6 hours, following which the compound of formula E is collected with an enantiomeric excess of at least 90%.

In some embodiments, the compound of formula D is dissolved in THF at a concentration of up to 0.2 g/mL, to which up to 1.5 equivalents of (1R)-(−)-10-camphorsulfonic acid is added at a temperature between 10-30° C. and held at that temperature for up to 3 hours before it is heated up to 50-75° C. and held at that temperature for up to 3 hours, following which the mixture is cooled to a temperature between 0-25° C. and held at that temperature for up to 6 hours, following which the compound of formula E is collected with an enantiomeric excess of at least 95%.

In some embodiments, the compound of formula D is dissolved in THF at a concentration of up to 0.2 g/mL, to which up to 1.5 equivalents of (1R)-(−)-10-camphorsulfonic acid is added at a temperature between 10-30° C. and held at that temperature for up to 3 hours before it is heated up to 50-75° C. and held at that temperature for up to 3 hours, following which the mixture is cooled to a temperature between 0-25° C. and held at that temperature for up to 6 hours, following which the compound of formula E is collected with an enantiomeric excess of at least 99%.

In some embodiments, the compound of formula D is recrystallized using 0.5 equivalents of (1R)-(−)-10-camphorsulfonic acid, and THE as a solvent. In some embodiments, the compound of formula D is dissolved in THF at a concentration of up to 0.1 g/mL, to which 0.5 equivalents of (1R)-(−)-10-camphorsulfonic acid is added. In some embodiments, the compound of formula D is dissolved in THF at a concentration of up to 0.1 g/mL, to which 0.5 equivalents of (1R)-(−)-10-camphorsulfonic acid is added at room temperature and held at that temperature for a period of time, at which point it is heated to an elevated temperature and held at that temperature for a period of time, following which it is cooled and held at that temperature for a period of time. In some embodiments, the compound of formula D is dissolved in THF at a concentration of up to 0.1 g/mL, to which 0.5 equivalents of (1R)-(−)-10-camphorsulfonic acid is added at 25° C. and held at that temperature for 1 hour before it is heated to 70° C. and held at that temperature for 1 hour, following which the mixture is cooled to 25° C. and held at that temperature for 1 hour, following which the compound of formula E is collected with an enantiomeric excess of at least 90%.

In some embodiments, the compound of formula D is dissolved in THF at a concentration of up to 0.1 g/mL, to which 0.5 equivalents of (1R)-(−)-10-camphorsulfonic acid is added at 25° C. and held at that temperature for 1 hour before it is heated to 70° C. and held at that temperature for 1 hour, following which the mixture is cooled to 25° C. and held at that temperature for 1 hour, following which the compound of formula E is collected with an enantiomeric excess of at least 95%.

In some embodiments, the compound of formula D is dissolved in THF at a concentration of up to 0.1 g/mL, to which 0.5 equivalents of (1R)-(−)-10-camphorsulfonic acid is added at 25° C. and held at that temperature for 1 hour before it is heated to 70° C. and held at that temperature for 1 hour, following which the mixture is cooled to 25° C. and held at that temperature for 1 hour, following which the compound of formula E is collected with an enantiomeric excess of at least 99%.

In step S2-3, the compound of formula E is alkylated with a compound of formula F to prepare a compound of formula G. Methods for N-alkylation of amines are well known to one of ordinary skill in the art. Exemplary reactions include those described in detail in Advanced Organic Chemistry: Reactions, Mechanisms, and Structure (3rd ed.), Jerry March, New York: Wiley, ISBN 0-471-85472-7, the entirety of which is incorporated herein by reference. In some embodiments, the compound of formula E and the compound of formula F are coupled to prepare the compound of formula G through reductive amination. In some embodiments, LG^{1B} is ═O. In some embodiments, LG^{1B}, taken with the carbon atom to which it is bound, an acetal. In some embodiments, PG^{1B}, taken with the nitrogen to which it is bound, is a cyclic imide. In some embodiments, PG^{1B} is absent and a triple bond is present between the corresponding nitrogen and the carbon to which the nitrogen is bound, such that the nitrogen and the carbon to which it is bound form a nitrile. In some embodiments, PG^{1B} is H and Boc.

In step S2-4, if R^{1B} is a benzimidazole protecting group and/or if PG^{1B}, taken with the nitrogen to which it is bound, is a cyclic imide, or if PG^{1B} includes any amino protecting group, or if PG^{1B} is absent and a triple bond is present between the corresponding nitrogen and the carbon to which the nitrogen is bound such that a nitrile is present, then the compound of formula G is deprotected to remove such protecting groups. Deprotection is understood to include reduction of the nitrile to an amino group. The protecting groups may be removed in a single step or in sequential steps. Deprotection methods of amine protecting groups and benzimidazole protecting groups are well known in the art and include those described in detail in Protecting Groups in Organic Synthesis. T. W. Greene and P. G. M. Wuts, 3^{rd} edition, John Wiley & Sons, 1999, the entirety of which is incorporated herein by reference. In some embodiments, the compound of formula G is deprotected using Raney Ni, H₂, and NH₃. In some embodiments, the compound of formula G is deprotected using HCl.

In one aspect, the present invention provides an alternative method to prepare the compound of formula G according to Scheme II′ below. The compound of formula G may then be used to prepare mavorixafor according to Scheme II.

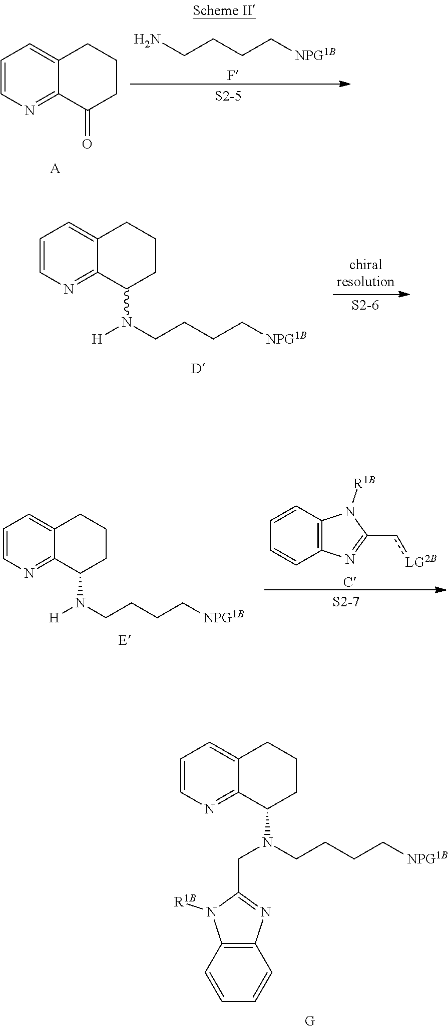

In one aspect, the present invention provides methods for preparing compounds of formulae F′, D′, E′, C′, and G according to the steps depicted in Scheme II′. In the compounds of the present formulae, R^{1B} is H, or a suitable benzimidazole protecting group; LG^{2B} is ═O, a suitable leaving group, or, taken with the carbon atom to which it is bound, an acetal; and PG^{1B} is any combination of two amino protecting groups, any single amino protecting group and H, or two H. PG^{1B} may also be, taken with the nitrogen to it is bound, a cyclic imide. Additionally, PG^{1B} may be absent and a triple bond is present between the corresponding nitrogen and the carbon to which the nitrogen is bound, such that the nitrogen and the carbon to which it is bound form a nitrile. It is understood in the art that a nitrile can function as a masked amino group. Thus, deprotection is understood to include reduction of the nitrile to an amino group.

In step S2-5, the compound of formula A is coupled with a compound of formula F′ to prepare a compound of formula D′. In some embodiments, the compound of formula A and the compound of formula F′ are coupled by reductive amination. Methods for reductive amination are well known in the art. In some embodiments, PG^{1B}, taken with the nitrogen to which it is bound, is a cyclic imide. In some embodiments, PG^{1B} is absent and a triple bond is present between the corresponding nitrogen and the carbon to which the nitrogen is bound, such that the nitrogen and the carbon to which it is bound form a nitrile. In some embodiments, PG^{1B} is H and Boc.

In step S2-6, the compound of formula D′ is purified to prepare the enantiomerically enriched compound E′. A compound is said to be enantiomerically enriched if the enantiomeric excess exceeds 70%. In some embodiments, the enantiomeric excess is greater than 80%. In some embodiments, the enantiomeric excess is greater than 90%. In some embodiments, the enantiomeric excess is greater than 95%. In some embodiments, the enantiomeric excess is greater than 97%. In some embodiments, the enantiomeric excess is greater than 99%. In some embodiments, the enantiomeric excess is greater than 99.9%.

In some embodiments, the compound of formula D′ is purified to prepare the enantiomerically enriched compound of formula E′ through recrystallization using a chiral acid. Chiral acids are well known in the art and include, but are not limited to(S)-2-(6-methoxynaphthalen-2-yl) propanoic acid, L-(+)-tartaric acid, (−)-di-p-toluoyl-L-tartaric acid, 2,3-di-O-para-toluoyl-D-tartaric acid, (−)-di-p-toluoyl-L-tartaric acid, (−)-dibenzoyl-L-tartaric acid, (+)-di-p-anisoyl-D-tartaric acid, L-malic acid, (S)-(+)-mandelic acid, (+)-camphoric acid, L-pyroglutamic acid, (S)-2-(6-methoxynaphthalen-2-yl) propanoic acid, N-Boc-D-proline, N-Boc-L-alanine, N-acetyl-L-tyrosine, N-acetyl-L-leucine, N-acetyl-L-proline, N-tosyl-L-proline, (1S)-(+)-10-camphorsulfonicacid, (1S)-(+)-10-camphorsulfonic acid, (1R)-(−)-10-camphorsulfonic acid, (S)-BNP acid, (S)-(+)-BNP, or (S)-Naproxen.

In some embodiments, the chiral acid is selected from one or more of L-(+)-tartaric acid, (−)-di-p-toluoyl-L-tartaric acid, 2,3-di-O-para-toluoyl-D-tartaric acid, (−)-dibenzoyl-L-tartaric acid, (+)-di-p-anisoyl-D-tartaric acid, (S)-(+)-mandelic acid, (+)-camphoric acid, L-pyroglutamic acid, (S)-2-(6-methoxynaphthalen-2-yl) propanoic acid, N-Boc-L-alanine, N-acetyl-L-tyrosine, N-acetyl-L-leucine, (1S)-(+)-10-camphorsulfonicacid, (1R)-(−)-10-camphorsulfonic acid, or (S)-(+)-BNP.

In some embodiments, the compound of formula D′ is purified to prepare the enantiomerically enriched compound of formula E′ through crystallization using one or more solvents selected from MeCN, THF, DCM, iPrOH, EA, EtOH or acetone.

In some embodiments, the compound of formula D′ is dissolved in solvent with the chiral acid at an elevated temperature before it is cooled down. In some embodiments, the compound of formula D′ is dissolved in solvent with the chiral acid at room temperature before it is heated to an elevated temperature, following which the mixture is cooled down. In some embodiments, the product collected from one crystallization is used to perform additional crystallizations.

In some embodiments, between 0.5 and 1.1 equivalents of chiral acid is used relative to the compound of formula D′. In some embodiments, between 1.0 and 2.5 equivalents of chiral acid is used relative to the compound of formula D′.

In some embodiments, the compound of formula D′ is initially dissolved in solvent at a concentration of up to 0.25 g/mL.

In some embodiments, the compound of formula D′ is crystallized using up to 1.5 equivalents of N-Boc-D-proline, and MeCN as a solvent. In some embodiments, the compound of formula D′ is dissolved in MeCN at a concentration of up to 0.15 g/mL, to which up to 1.5 equivalents of N-Boc-D-proline is added. In some embodiments, the compound of formula D′ is dissolved in MeCN at a concentration of up to 0.15 g/mL, to which up to 1.5 equivalents of N-Boc-D-proline is added at room temperature and held at that temperature for a period of time, at which point it is heated to an elevated temperature and held at that temperature for a period of time, following which it is cooled and held at that temperature for a period of time. In some embodiments, the compound of formula D′ is dissolved in MeCN at a concentration of up to 0.15 g/mL, to which up to 1.5 equivalents of N-Boc-D-proline is added at a temperature between 10-25° C. and held at that temperature for up to 3.0 hours before it is heated to 60-82° C. and held at that temperature for up to 3.0 hour, following which the mixture is cooled to a temperature between 0-25° C. and held at that temperature for up to 20 hours, following which the compound of formula E′ is collected with an enantiomeric excess of at least 90%.

In some embodiments, the compound of formula D′ is dissolved in MeCN at a concentration of up to 0.15 g/mL, to which up to 1.5 equivalents of N-Boc-D-proline is added at a temperature between 10-25° C. and held at that temperature for up to 3.0 hours before it is heated to 60-82° C. and held at that temperature for up to 3.0 hour, following which the mixture is cooled to a temperature between 0-25° C. and held at that temperature for up to 20 hours, following which the compound of formula E′ is collected with an enantiomeric excess of at least 95%

In some embodiments, the compound of formula D′ is dissolved in MeCN at a concentration of up to 0.15 g/mL, to which up to 1.5 equivalents of N-Boc-D-proline is added at a temperature between 10-25° C. and held at that temperature for up to 3.0 hours before it is heated to 60-82° C. and held at that temperature for up to 3.0 hour, following which the mixture is cooled to a temperature between 0-25° C. and held at that temperature for up to 20 hours, following which the compound of formula E′ is collected with an enantiomeric excess of at least 99%.

In some embodiments, the compound of formula D′ is crystallized using 0.5 equivalents of N-Boc-D-proline, and MeCN as a solvent. In some embodiments, the compound of formula D′ is dissolved in MeCN at a concentration of up to 0.07 g/mL, to which 0.5 equivalents of N-Boc-D-proline is added. In some embodiments, the compound of formula D′ is dissolved in MeCN at a concentration of up to 0.07 g/mL, to which 0.5 equivalents of N-Boc-D-proline is added at room temperature and held at that temperature for a period of time, at which point it is heated to an elevated temperature and held at that temperature for a period of time, following which it is cooled and held at that temperature for a period of time. In some embodiments, the compound of formula D′ is dissolved in MeCN at a concentration of up to 0.07 g/mL, to which 0.5 equivalents of N-Boc-D-proline is added at a temperature between 20-25° C. and held at that temperature for 0.5 hours before it is heated to 75-80° C. and held at that temperature for 0.5 hours, following which the mixture is cooled to a temperature between 20-25° C. and held at that temperature for 16 hours, following which the compound of formula E′ is collected with an enantiomeric excess of at least 90%.

In some embodiments, the compound of formula D′ is dissolved in MeCN at a concentration of up to 0.07 g/mL, to which 0.5 equivalents of N-Boc-D-proline is added at a temperature between 20-25° C. and held at that temperature for 0.5 hours before it is heated to 75-80° C. and held at that temperature for 0.5 hours, following which the mixture is cooled to a temperature between 20-25° C. and held at that temperature for 16 hours, following which the compound of formula E′ is collected with an enantiomeric excess of at least 95%.

In some embodiments, the compound of formula D′ is dissolved in MeCN at a concentration of up to 0.07 g/mL, to which 0.5 equivalents of N-Boc-D-proline is added at a temperature between 20-25° C. and held at that temperature for 0.5 hours before it is heated to 75-80° C. and held at that temperature for 0.5 hours, following which the mixture is cooled to a temperature between 20-25° C. and held at that temperature for 16 hours, following which the compound of formula E′ is collected with an enantiomeric excess of at least 99%.

In step S2-7, the compound of formula E′ is alkylated with the compound of formula C′ to prepare the compound of formula G. Methods for N-alkylation of amines are well known to one of ordinary skill in the art. Exemplary reactions include those described in detail in Advanced Organic Chemistry: Reactions, Mechanisms, and Structure (3rd ed.), Jerry March, New York: Wiley, ISBN 0-471-85472-7, the entirety of which is incorporated herein by reference. In some embodiments, the compound of formula E′ is coupled with the compound of formula C′ to prepare the compound of formula G using reductive amination. In some embodiments, LG^{2B} is ═O. In some embodiments, LG^{2B} is, taken with the carbon atom to which it is bound, an acetal. In some embodiments, R^{1B} is CH₂OCH₂Ph.

In one aspect, the present invention provides a method of preparing mavorixafor, or a pharmaceutically acceptable salt thereof, as set forth in Scheme III below.

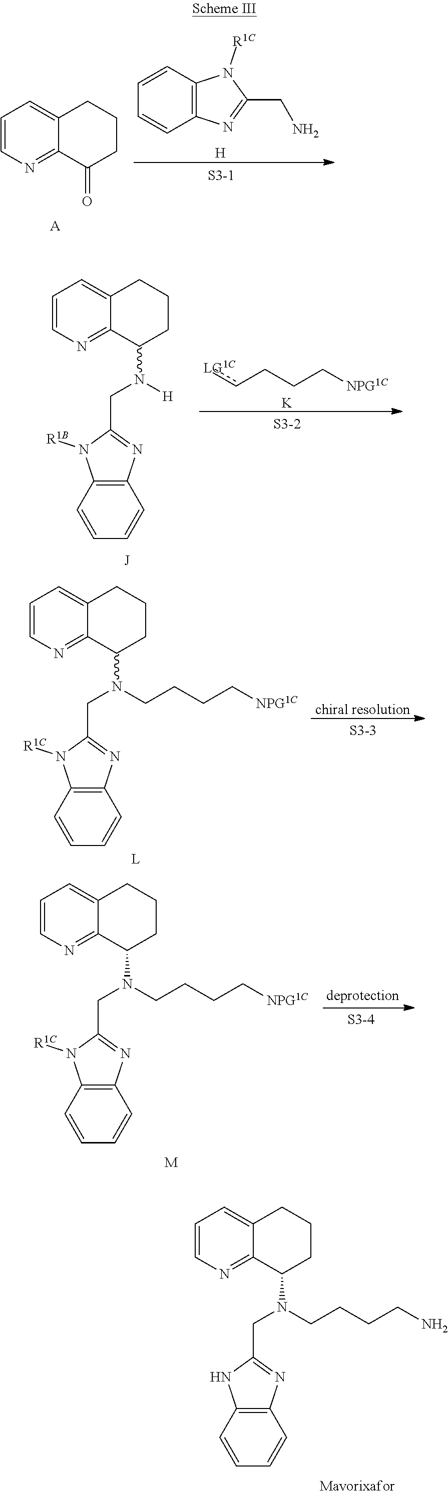

In one aspect, the present invention provides methods for preparing compounds of formulae H, J, K, L, M, and mavorixafor according to the steps depicted in Scheme III. In the compounds of the present formulae, R¹C is H, or a suitable benzimidazole protecting group; LG^{1C} is ═O, a suitable leaving group, or, taken with the carbon atom to which it is bound, an acetal; and PG^{1C} is any combination of two amino protecting groups, a single amino protecting group and H, or two H. PG^{1C} may also be, taken with the nitrogen to which it is bound, a cyclic imide. Additionally, PG^{1C} may be absent and a triple bond present between the corresponding nitrogen and the carbon to which the nitrogen is bound, such that the nitrogen and the carbon to which it is bound form a nitrile.

In step S3-1, the compound of formula A is coupled with the compound of formula H to prepare the compound of formula J. In some embodiments, the compound of formula A and the compound of formula H are coupled to prepare the compound of formula J through reductive amination. Methods for reductive amination are well known in the art. In some embodiments, R^{1C} is H.

In step S3-2, the compound of formula J is alkylated with a compound of formula K to prepare a compound of formula L. Methods for N-alkylation of amines are well known to one of ordinary skill in the art. Exemplary reactions include those described in detail in Advanced Organic Chemistry: Reactions, Mechanisms, and Structure (3rd ed.), Jerry March, New York: Wiley, ISBN 0-471-85472-7, the entirety of which is incorporated herein by reference. In some embodiments, LG^{1C} is ═O. In some embodiments, LG^{1C} is, taken with the carbon atom to which it is bound, an acetal. In some embodiments the compound of formula J is coupled with the compound of formula K to prepare a compound of formula L through reductive amination. In some embodiments, PG^{1C} is H and Boc. In some embodiments, PG^{1C} is a cyclic imide. In some embodiments, PG^{1C} is absent and a triple bond is present between the corresponding nitrogen and the carbon to which the nitrogen is bound, such that the nitrogen and the carbon to which it is bound form a nitrile.

In step S3-3, the compound of formula L is purified to prepare the enantiomerically enriched compound M. A compound is said to be enantiomerically enriched if the enantiomeric excess exceeds 70%. In some embodiments, the enantiomeric excess is greater than 80%. In some embodiments, the enantiomeric excess is greater than 90%. In some embodiments, the enantiomeric excess is greater than 95%. In some embodiments, the enantiomeric excess is greater than 97%. In some embodiments, the enantiomeric excess is greater than 99%. In some embodiments, the enantiomeric excess is greater than 99.9%.

In some embodiments, the compound of formula L is purified to prepare the enantiomerically enriched compound of formula M using chiral HPLC. In some embodiments, the compound of formula L is purified to prepare the enantiomerically enriched compound of formula M using chiral HPLC in which a supercritical fluid is used as the mobile phase. In some embodiments, the compound of formula L is purified to prepare the enantiomerically enriched compound of formula M using chiral HPLC in which a supercritical fluid mobile phase composed of [CO₂-EtOH (0.1% NH₃H₂O)] is used. In some embodiments, the compound of formula L is purified to prepare the enantiomerically enriched compound of formula M using chiral HPLC in which a supercritical fluid mobile phase composed of [CO₂-EtOH (0.1% NH₃H₂O)] is used and the compound of formula M is isolated with at least 90% enantiomeric excess. In some embodiments, the compound of formula L is purified to prepare the enantiomerically enriched compound of formula M using chiral HPLC in which a supercritical fluid mobile phase composed of [CO₂-EtOH (0.1% NH₃H₂O)] is used and the compound of formula M is isolated with at least 95% enantiomeric excess. In some embodiments, the compound of formula L is purified to prepare the enantiomerically enriched compound of formula M using chiral HPLC in which a supercritical fluid mobile phase composed of [CO₂-EtOH (0.1% NH₃H₂O)] is used and the compound of formula M is isolated with at least 99% enantiomeric excess.

In step S3-4, if R¹C is a benzimidazole protecting group and/or if PG^{1C}, taken with the nitrogen atom to which it is bound, is a cyclic imide, or if PG^{1C} includes any amino protecting group, or if PG^{1C} is absent and a triple bond is present between the corresponding nitrogen and the carbon to which the nitrogen is bound such that a nitrile is present, then the compound of formula M is deprotected to remove such protecting groups. Deprotection is understood to include reduction of the nitrile to an amino group. The protecting groups may be removed in a single step or in sequential steps. Deprotection methods of amine protecting groups and benzimidazole protecting groups are well known in the art and include those described in detail in Protecting Groups in Organic Synthesis, T. W. Greene and P. G. M. Wuts, 3rd edition, John Wiley & Sons, 1999, the entirety of which is incorporated herein by reference. In some embodiments, the deprotection is performed using HCl.

In one aspect, the present invention provides an alternative method to prepare the compound of formula L according to Scheme III′ below. The compound of formula L may then be used to prepare mavorixafor according to Scheme III.

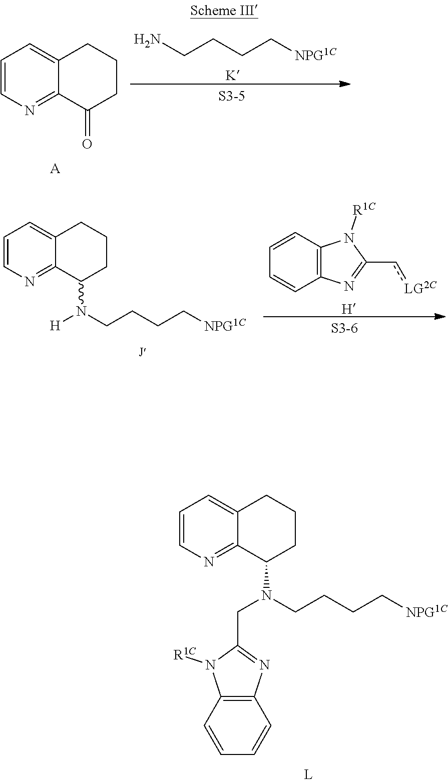

In one aspect, the present invention provides methods for preparing compounds of formulae K′, J′, H′, and L according to the steps depicted in Scheme III′. In the compounds of the present formulae, R¹C is H, or a suitable benzimidazole protecting group; LG^{2C} is ═O, a suitable leaving group, or, taken with the carbon atom to which it is bound, an acetal; and PG^{1C} is any combination of two amino protecting groups, any single amino protecting group and H, or two H. PG^{1C} may also be, taken with the nitrogen to which it is bound, a cyclic imide. Additionally, PG^{1C} may be absent and a triple bond present between the corresponding nitrogen and the carbon to which the nitrogen is bound, such that the nitrogen and the carbon to which it is bound form a nitrile. It is understood in the art that a nitrile can function as a masked amino group. Thus, deprotection is understood to include reduction of the nitrile to an amino group.

In step S3-5, the compound of formula A is coupled to a compound of formula K′ to prepare a compound of formula J′. In some embodiments, the compound of formula A is coupled to a compound of formula K′ to prepare a compound of formula J′ using reductive amination. Methods of reductive amination are well known in the art. In some embodiments, PG^{1C} is H and Boc. In some embodiments, PG^{1C} is a cyclic imide. In some embodiments, PG^{1C} is absent and a triple bond is present between the corresponding nitrogen and the carbon to which the nitrogen is bound, such that the nitrogen and the carbon to which it is bound form a nitrile.

In step S3-6, the compound of formula J′ is alkylated with the compound of formula H′ to prepare the compound of formula M. Methods for N-alkylation of amines are well known to one of ordinary skill in the art. Exemplary reactions include those described in detail in Advanced Organic Chemistry: Reactions, Mechanisms, and Structure (3rd ed.), Jerry March, New York: Wiley, ISBN 0-471-85472-7, the entirety of which is incorporated herein by reference. In some embodiments, R¹C is H. In some embodiments, LG^{2C} is ═O. In some embodiments, LG^{2C} is, taken with the carbon atom to which it is bound, an acetal.

In one aspect, the present invention provides a method of preparing mavorixafor, or a pharmaceutically acceptable salt thereof, as set forth in Scheme IV below.

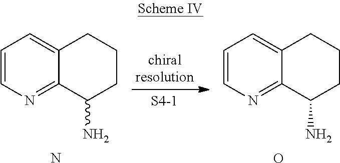

In one aspect, the present invention provides methods for preparing the compound of formula O according to the step depicted in Scheme IV.

In step S4-1, the compound of formula N is purified to prepare the enantiomerically enriched compound O. A compound is said to be enantiomerically enriched if the enantiomeric excess exceeds 70%. In some embodiments, the enantiomeric excess is greater than 80%. In some embodiments, the enantiomeric excess is greater than 90%. In some embodiments, the enantiomeric excess is greater than 95%. In some embodiments, the enantiomeric excess is greater than 97%. In some embodiments, the enantiomeric excess is greater than 99%. In some embodiments, the enantiomeric excess is greater than 99.9%. In some embodiments, the compound of formula N is purified to prepare the enantiomerically enriched compound of formula O by chiral HPLC. In some embodiments, the compound of formula N is purified to prepare the enantiomerically enriched compound of formula O by crystallization with a chiral acid.

### EXEMPLIFICATION

### Example 1: First Synthesis of Mavorixafor

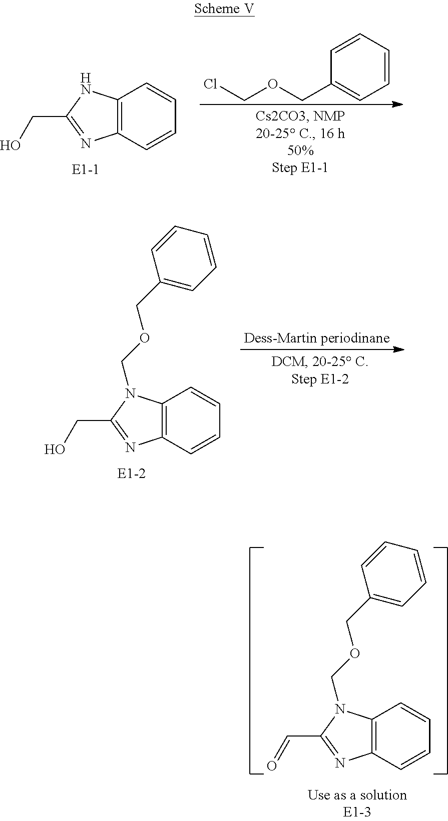

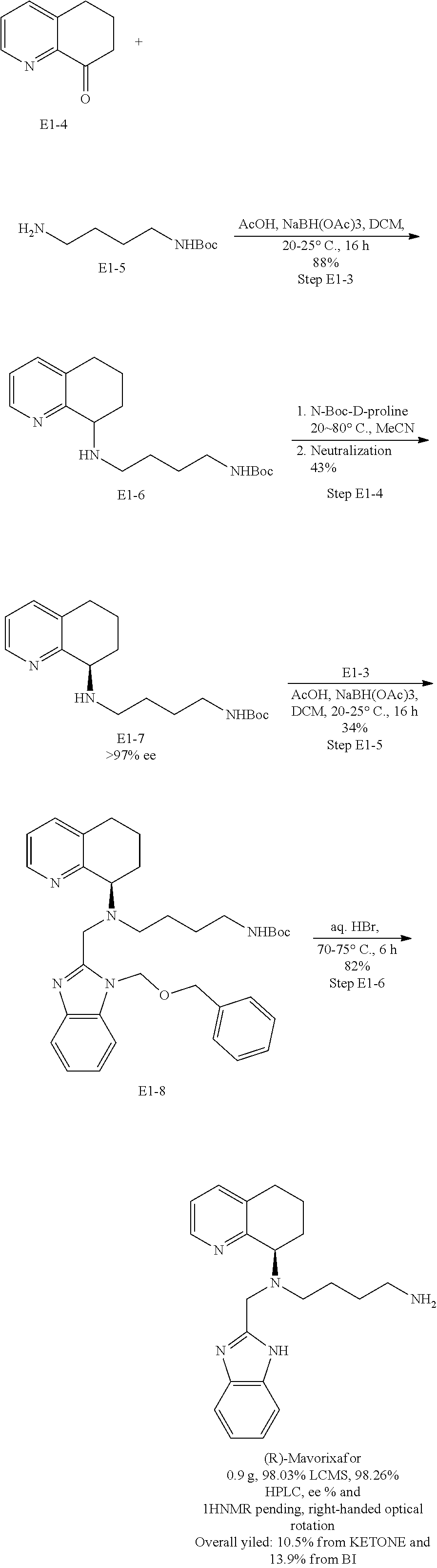 [SMILES: O=C1CCCc2cccnc21 ; CC(C)(C)OC(=O)NCCCCN ; CC(C)(C)OC(=O)NCCCCNC1CCCc2cccnc21 ; CC(C)(C)OC(=O)NCCCCN[C@@H]1CCCc2cccnc21 ; CC(C)(C)OC(=O)NCCCCN(Cc1nc2ccccc2n1COCc1ccccc1)[C@@H]1CCCc2cccnc21 ; NCCCCN(Cc1nc2ccccc2[nH]1)[C@@H]1CCCc2cccnc21]

### Step E1-1: Protection of E1-1

To a solution of (1H-benzo[d]imidazol-2-yl) methanol (E1-1, 40 g, 270.27 mmol) in NMP (400 mL) was added Cs₂CO₃ (123.3 g, 378.39 mmol) and ((chloromethoxy)methyl)benzene (BOMCl, 50.7 g, 323.96 mmol) at 0-5° C., and then the mixture was stirred at 20-25° C. for 16 h. LCMS analysis indicated 60% product formation with 15% of the starting material remaining. The mixture was poured into EtOAc (450 mL) and water (200 mL). The organic phase was collected and the aqueous phase was extracted with the EtOAc (300 mL×2). The combined organic phase was washed with H₂O (200 mL×4) and then concentrated in vacuum to give a residue, which was purified by column chromatography (EA:PE=1:2 to EA) to give (1-((benzyloxy)methyl)-1H-benzo[d]imidazol-2-yl) methanol (E1-2, 30 g, 42%) as a light-yellow solid.

### Step E1-2: Oxidation of E1-2

To a solution of (1-((benzyloxy)methyl)-1H-benzo[d]imidazol-2-yl) methanol (E1-2, 15 g, 56.00 mmol) in DCM (300 mL) was added Dess-Martin periodinane (47.5 g, 112 mmol), then the mixture was stirred at 20-25° C. for 3 h. LCMS analysis indicated 50% product formation, along with other unknown peaks. The mixture was filtered and the filtrate was collected directly for the next step.

### Step E1-3: Coupling of E1-4 and E1-5

To a solution of 6,7-dihydroquinolin-8 (5H)-one (E1-4, 20 g, 135.89 mmol) in DCM (500 mL) was added (tert-butyl (4-aminobutyl) carbamate (E1-5, 25.6 g, 135.89 mmol), NaBH(OAc)₃ (37.48 g, 176.66 mmol) and AcOH (0.82 g, 13.59 mmol) at 20-25° C., and then the mixture was stirred at 20-25° C. for 16 h. After completion of the reaction as indicated by LCMS, the mixture was washed with aq. NaHCO₃ (200 mL×1), brine (200 mL×1), dried over Na₂SO₄, filtered and concentrated in vacuum to afford tert-butyl (4-((5,6,7,8-tetrahydroquinolin-8-yl)amino)butyl) carbamate (E1-6, 42 g, 99%) as a brown solid.

Step E1-4: Chiral resolution of E1-6

To a solution of N-Boc-D-proline (6.74 g, 31.35 mmol) in MeCN (150 mL) was added tert-butyl (4-((5,6,7,8-tetrahydroquinolin-8-yl)amino)butyl) carbamate (E1-6, 10.00 g, 31.35 mmol) at 20-25° C. The mixture was stirred at 20-25° C. for 0.5 h and heated to 75-80° C. for 0.5 h to give a clear solution. The solution was cooled to 20-25° C. in the oil bath and left for 16 h at 20-25° C. The solid was collected by filtration and washed with MeCN (20 mL×2). The cake was added into MTBE (300 mL) and aq. Na₂CO₃ (10 wt %, 100 mL) at 20-25° C. The organic phase was collected and washed with H₂O (200 mL×1), dried over Na₂SO₄, and concentrated in vacuum to afford tert-butyl (R)-(4-((5,6,7,8-tetrahydroquinolin-8-yl)amino)butyl) carbamate (E1-7, 4.3 g, 43%) as a white solid.

### Step E1-5: Coupling of E1-3 and E1-7

To a solution of 1-((benzyloxy)methyl)-1H-benzo[d]imidazole-2-carbaldehyde (E1-3 prepared from 15 g of E1-2, 56.00 mmol) in DCM (300 mL) was added tert-butyl (R)-(4-((5,6,7,8-tetrahydroquinolin-8-yl)amino)butyl) carbamate (E1-7, 8.9 g, 28 mmol), NaBH(OAc)₃ (17.8 g, 84 mmol) and AcOH (0.16 g, 2.8 mmol) at 20-25° C., and then the mixture was stirred at 20-25° C. for 16 h. After completion of the reaction as indicated by LCMS, the mixture was washed with aq. NaHCO₃ (200 mL×1), brine (200 mL×1), and dried over Na₂SO₄. After filtering off the Na₂SO₄, the organic phase was concentrated in vacuum to give a residue, which was purified by column chromatography (DCM:MeOH=20:1) to give tert-butyl (R)-(4-(((1-((benzyloxy)methyl)-1H-benzo[d]imidazol-2-yl)methyl) (5,6,7,8-tetrahydroquinolin-8-yl)amino)butyl) carbamate ((R)-E1-8, 5.3 g, 34%) as a brown oil.

### Step E1-6: Deprotection of E1-8

To a solution of aq. HBr (44 wt %, 18 mL) was added 1H-benzo[d]imidazol-2-yl)methyl) (5,6,7,8-tetrahydroquinolin-8-yl)amino)butyl) carbamate (E1-8, 1.8 g, 54.42 mmol) at 20-25° C. and then the mixture was stirred at 70-75° C. for 16 h. After completion of the reaction as indicated by LCMS, activated carbon (0.36 g) was added to the mixture. After stirring for an extra 0.5 h at 20-25° C., the mixture was filtered and washed with hot water (10 mL×2, 60° C.). The filtrate was washed with toluene (20 mL×3) and then the organic phase was concentrated in vacuum to give a residue (~5 g). The residue was diluted with water (10 mL) and then added into NH₃·H₂O (10 wt %, 18 mL) at 0-5° C. with stirring. The aqueous phase was decanted and the semisolid was washed with water (2 mL×2), dried under high vacuum (oil pump) at 20-25° C. to give (R)-N-((1-(ethoxymethyl)-1H-benzo[d]imidazol-2-yl)methyl)-5,6,7,8-tetrahydroquinolin-8-amine ((R)-Mavorixafor, 0.90 g, 82%) as yellow solid.

### Example 2: Second Synthesis of Mavorixafor

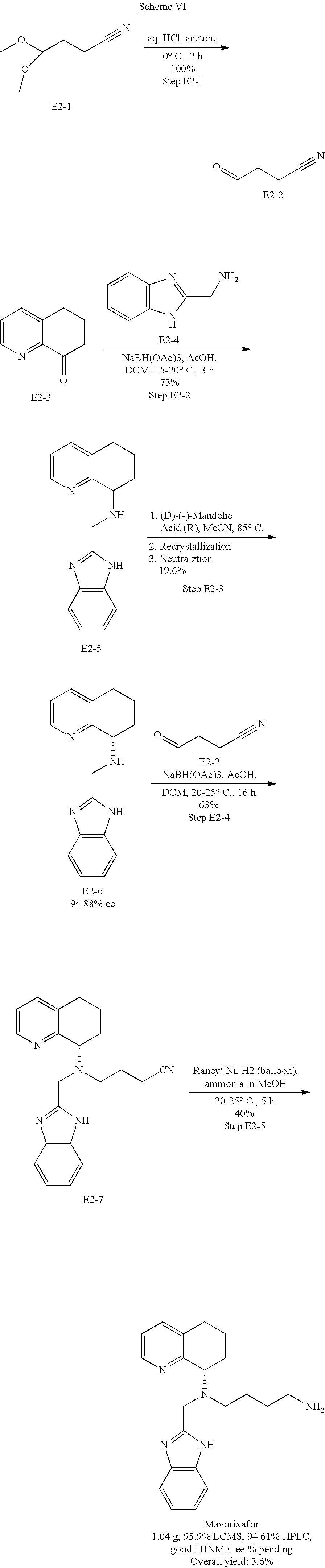

### Step E2-1: Deprotection of E2-1

To a solution of 4,4-dimethoxybutanenitrile (E2-1, 14 g, 7.742 mmol) in acetone (560 mL) was added 6 N HCl (210 mL) at 0° C., and then the mixture was stirred at 0° C. for 2 h. After completion of the reaction as indicated by TLC, the mixture was extracted with DCM (200 mL×5). The organic layer was washed with brine, dried over Na₂SO₄, filtered, and concentrated in vacuum to give 4-oxobutanenitrile (E2-2, 9 g, 100%), which was used in the next step without further purification.

### Step E2-2: Coupling of E2-3 and E2-4

To a solution of 6,7-dihydroquinolin-8 (5H)-one (E2-3, 30 g, 0.20 mol) in DCM (300 mL) was added (1H-benzo[d]imidazol-2-yl) methanamine (E2-4, 42 g, 0.29 mmol) and NaBH(OAc)₃ (86.46 g, 0.4 mol) at 20-25° C., and then the mixture was stirred at 20-25° C. for 2 h. After completion of the reaction as indicated by LCMS, the mixture was washed with aq. NaHCO₃ (300 mL×1), brine (300 mL×1), dried over Na₂SO₄, filtered and concentrated in vacuum to give a residue. The residue was purified by column chromatography (DCM:MeOH=100:1) to give a brown product. The brown product was then resuspended with toluene (80 mL) at 0-5° C. for 2 h, and the solid was collected by filtration and dried under reduced pressure by oil pump at 20-25° C. to give N-((1H-benzo[d]imidazol-2-yl)methyl)-5,6,7,8-tetrahydroquinolin-8-amine (E2-5, 42 g, 74%) as a brown solid.

Step E2-3: Chiral resolution of E2-5

To a solution of N-((1H-benzo[d]imidazol-2-yl)methyl)-5,6,7,8-tetrahydroquinolin-8-amine (E2-5, 81 g, 0.29 mol) in MeCN (800 mL) was added R-(−)-Mandelic Acid (44.29 g, 0.29 mol) at 20-25° C., and then the mixture was heated to 80-85° C. and stirred at 80-85° C. for 0.5 h. The mixture was gradually cooled to 20-25° C. during 2 h and stirred at 20-25° C. for 3 h. The solid was collected by filtration. The wet solid was added to MeCN (800 mL) and then the mixture was heated to 80-85° C. and stirred at 80-85° C. for 0.5 h. The mixture was gradually cooled to 20-25° C. during 2 h and stirred at 20-25° C. for 3 h, and the solid was collected by filtration. The wet solid was added into MeCN (600 mL) and then the mixture was heated to 80-85° C. and stirred at 80-85° C. for 0.5 h. The mixture was gradually cooled to 20-25° C. during 2 h and stirred at 20-25° C. for 3 h. The solid was collected by filtration. The wet solid was suspended between DCM (300 mL) and water (200 mL), and neutralized with solid NaOH to a pH of 10-11. The organic phase was collected and washed with brine (100 mL), dried over Na₂SO₄, filtered and concentrated to give (S)-N-((1H-benzo[d]imidazol-2-yl)methyl)-5,6,7,8-tetrahydroquinolin-8-amine (E2-6, 13.6 g, 19.6%) with 94.88% ee.

### Step E2-4: Coupling of E2-2 and E2-6

To a solution of (S)-N-((1H-benzo[d]imidazol-2-yl)methyl)-5,6,7,8-tetrahydroquinolin-8-amine (E2-6, 4 g, 14.4 mmol) in MeOH (40 mL) was added 4-oxobutanenitrile (E2-2, 2.03 g, 24 mmol), AcOH (0.087 g, 1.44 mmol) and NaBH₃CN (1.8 g, 28.8 mmol) at 0-5° C., and then the mixture was stirred at 20-25° C. overnight. After completion of the reaction as indicated by LCMS, the mixture was diluted with water (80 mL) and extracted with DCM (80 mL×3). The organic layer was washed with aq. NaHCO₃ (60 mL×1), brine (60 mL×1), dried over Na₂SO₄, filtered and concentrated in vacuum to give a residue, which was purified by column chromatography (DCM:MeOH=100:1 to 5:1) to give (S)-4-(((1H-benzo[d]imidazol-2-yl)methyl) (5,6,7,8-tetrahydroquinolin-8-yl)amino)butanenitrile (E2-7, 3.13 g, 63%) as white solid.

### Step E2-5: Reduction of E2-7

To a solution of ((S)-4-(((1H-benzo[d]imidazol-2-yl)methyl) (5,6,7,8-tetrahydroquinolin-8-yl)amino)butanenitrile (E2-7, 2.5 g, 43 mmol) in 7 M NH₃ in MeOH (25 mL) was added Raney Ni (3 g). The mixture was evacuated and backfilled with H₂ three times, then was stirred under H₂ (balloon) at 20-50° C. for 5 h. After completion of the reaction as indicated by LCMS, the mixture was filtered and the filter cake was washed with MeOH (20 mL×2). The filtrate was concentrated in vacuum to give a residue, which was purified by silica gel column chromatography (THF:MeOH:NH₄OH=90:10:1) to give (S)-N-((1-(ethoxymethyl)-1H-benzo[d]imidazol-2-yl)methyl)-N-((S)-1-(4-methoxyphenyl)-ethyl)-5,6,7,8-tetrahydroquinolin-8-amine (Mavorixafor, 1.04 g, 40%) as white solid.

### Example 3: Third Synthesis of Mavorixafor

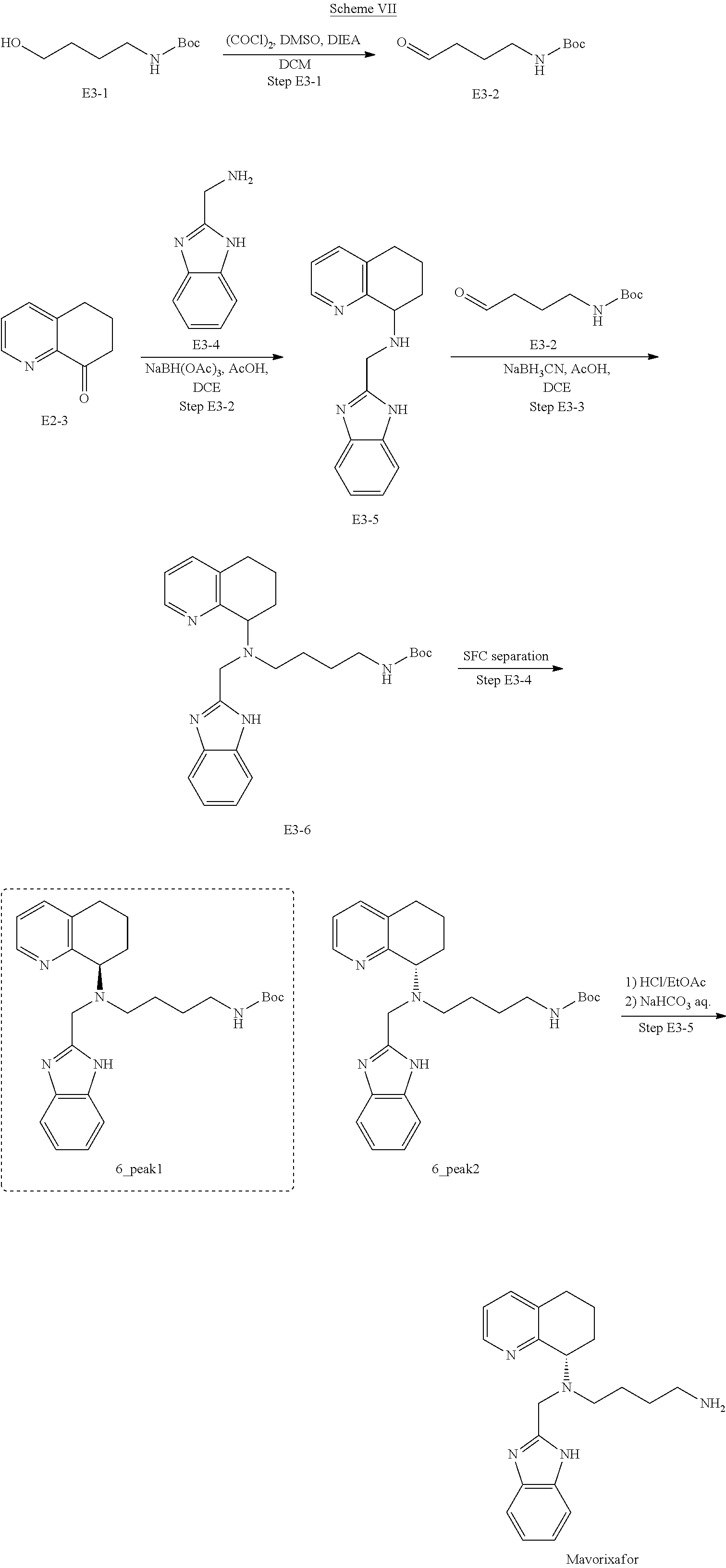

### Step E3-1: Oxidation of E3-1

A mixture of oxalyl chloride (7.98 g, 62.9 mmol, 5.50 mL, 1.19 eq) in DCM (25.0 mL) was added to a solution of DMSO (9.00 g, 115 mmol, 9.00 mL, 2.18 eq) in DCM (100 mL) over 30 min at-65° C., and the mixture was stirred for another 15 min. Then a solution of compound E3-1 (10.0 g, 52.8 mmol, 1.00 eq) in DCM (30.0 mL) was added over 30 min, and stirred for 15 min. DIEA (20.5 g, 159 mmol, 27.6 mL, 3.00 eq) was added over 5 min. After stirring of 10 min, the mixture was warmed to 0° C. and stirred at 0° C. for 30 min. TLC (Petroleum ether:Ethyl acetate=1:1) indicated all of compound E3-1 was consumed and many new spots were detected. The reaction mixture was diluted with H₂O (200 mL) at 0° C. and stirred at 0° C. for 5 min, and then extracted with MTBE (150 mL*3). The combined organic layers were washed with H₂O (100 mL*2), dried over Na₂SO₄, filtered and concentrated under reduced pressure to give a residue. Compound E3-2 (11.0 g, crude) was obtained as a yellow oil, confirmed by crude 1H NMR. 1H NMR (400 MHZ, CDCl₃): δ 7.74 (t, J=1.2 Hz, 1H), 4.60 (br s, 1H), 3.16 (d, J=6.4 Hz, 2H), 2.51 (t, J=6.0 Hz, 2H), 1.82 (q, J=7.2 Hz, 2H), 1.44 (s, 9H).

### Step E3-2: Coupling of E3-3 and E3-4

A mixture of compound E3-3 (6.50 g, 44.2 mmol, 1.00 eq), compound E3-4 (6.50 g, 44.2 mmol, 1.00 eq) and AcOH (3.98 g, 66.3 mmol, 3.79 mL, 1.50 eq) in DCE (65.0 mL) was stirred at 20° C. for 0.5 hr, and then NaBH(OAc)₃ (14.0 g, 66.3 mmol, 1.50 eq) was added and the mixture was stirred at 20° C. for another 10 hrs. LC-MS showed ~77% formation of a peak corresponding to the desired mass. The mixture was quenched with sat. NaHCO₃ (150 mL) extracted with DCM (150 mL*3). The organic layers were washed with H₂O (150 mL*2), dried over Na₂SO₄, filtered and concentrated. The crude product was triturated with PE:EA (1:1, 60.0 mL), the solid was filtered and collected, and then concentrated under reduced pressure. Compound E3-5 (6.50 g, 21.1 mmol, 47.8% yield, 90.4% purity) was obtained as a yellow solid, and the identity was confirmed by 1H NMR and LC-MS. LC-MS: (M+H)⁺: 279.1; 1H NMR (400 MHZ, CDCl₃): δ 8.55 (d, J=4.0 Hz, 1H), 7.60 (dd, J₁=6.0 Hz, J₂=2.4 Hz, 2H), 4.99 (d, J=8.0 Hz, 1H), 7.25-7.19 (m, 3H), 4.43-4.33 (m, 2H), 3.91 (t, J=6.0 Hz, 1H), 2.89-2.72 (m, 2H), 2.13-2.10 (m, 1H), 2.00-1.96 (m, 1H), 1.84-1.74 (m, 2H).

### Step E3-3: Coupling of E3-2 and E3-5

A mixture of compound E3-5 (9.18 g, 33.0 mmol, 1.00 eq), compound E3-2 (9.26 g, 49.5 mmol, 1.50 eq) and AcOH (396 mg, 6.60 mmol, 378 μL, 0.200 eq) in DCE (100 mL) was stirred at 20° C. for 10 hrs, and then NaBH₃CN (4.15 g, 67.0 mmol, 2.00 eq) was added at 0° C. and stirred at 20° C. for another 2 hrs. LC-MS showed ~39% formation of a peak corresponding to the desired mass. The mixture was diluted with sat. NaHCO₃ (150 mL) and extracted with DCM (150 mL*3). The combined organic layers were washed with brine (100 mL*1), dried over Na₂SO₄, filtered and concentrated under reduced pressure. The residue was purified by prep-HPLC (column: Phenomenex luna C18 (250*70 mm, 10 μm); mobile phase: [water (FA)-ACN]; gradient: 13%-35% B over 15 min). Compound E3-6 (6.50 g, 12.6 mmol, 38.2% yield, 96.1% purity) was obtained as a yellow gum. The product was confirmed by ¹H NMR and LC-MS. LC-MS: (M+H)⁺: 450.4; ¹H NMR (400 MHZ, CDCl₃): δ 8.73 (d, J=5.2 Hz, 1H), 7.68-7.65 (m, 2H), 7.61 (d, J=12.6 Hz, 1H), 7.34-7.30 (m, 3H), 4.71 (b rs, 1H), 4.41-4.26 (m, 2H), 4.13 (dd, J₁=10.0 Hz, J₂=6.4 Hz, 1H), 2.99-2.86 (m, 3H), 2.80-2.70 (m, 2H), 2.56-2.50 (m, 1H), 2.31-2.28 (m, 1H), 2.09-1.99 (m, 1H), 2.00-1.97 (m, 1H), 1.47-1.44 (m, 1H), 1.51-1.44 (m, 2H), 1.40 (s, 9H), 1.37-1.33 (m, 2H).

Step E3-4: SFC separation of E3-6

Compound E3-6 was purified by prep-HPLC (column: DAICEL CHIRALPAK AD-H (250 mm*30 mm, 5 μm); mobile phase: [CO₂-EtOH (0.1% NH₃H₂O)]; B %: 30%, isocratic elution mode). Compound 6_peak 1 (2.70 g, 6.01 mmol, 41.5% yield, 100% purity) was obtained as a yellow gum, and was confirmed by ¹H NMR, LC-MS, and SFC. Compound 6_peak 2 (3.00 g, 6.46 mmol, 44.7% yield, 96.8% purity) was obtained as a yellow gum, and was confirmed by ¹H NMR, LC-MS, and SFC. Analytical data of 6_peak 1: ¹H NMR (400 MHz, CDCl₃): δ 8.59 (d, J=3.6 Hz, 1H), 7.59 (s, 2H), 7.44 (d, J=8.0 Hz, 1H), 7.23-7.15 (m, 3H), 4.87 (b rs, 1H), 4.17-4.03 (m, 3H), 2.98-2.80 (m, 3H), 2.76-2.71 (m, 2H), 2.60-2.54 (m, 1H), 2.23-2.20 (m, 1H), 2.07-2.04 (m, 1H), 1.97-1.88 (m, 1H), 1.72-1.68 (m, 1H), 1.52-1.47 (m, 1H), 1.41 (s, 9H), 1.38-1.34 (m, 2H); SFC chiral purity: 100%; LC-MS (M+H)⁺: 450.3. Analytical data of 6_peak2: ¹H NMR (400 MHZ, CDCl₃): δ 8.65 (d, J=4.4 Hz, 1H), 8.43 (s, 1H), 7.63 (dd, J₁=6.0 Hz, J₂=3.2 Hz, 2H), 7.51 (d, J=7.6 Hz, 1H), 7.28-7.21 (m, 2H), 4.59 (b rs, 1H), 4.22 (q, J=12.8 Hz, 2H), 4.07 (dd, J₁=10.0 Hz, J₂=6.4 Hz, 1H), 2.95-2.88 (m, 3H), 2.78-2.71 (m, 2H), 2.59-2.52 (m, 1H), 2.27-2.23 (m, 1H), 2.09-2.05 (m, 1H), 1.99-1.93 (m, 1H), 1.73-1.69 (m, 1H), 1.48-1.42 (m, 2H), 1.40 (s, 9H), 1.36-1.33 (m, 2H); SFC: chiral purity: 98.2%; LC-MS (M+H)⁺: 450.4

Step E3-5: Deprotection of 6_peak2

A mixture of compound 6_peak 2 (3.00 g, 6.67 mmol, 1.00 eq) and HCl/EtOAc (4 M, 10.0 mL, 5.99 eq) in DCM (30.0 mL) was stirred at 20° C. for 2 hrs. LC-MS showed ~90% formation of a peak with the desired mass. The mixture was concentrated to remove solvent. The mixture was diluted with H₂O (30.0 mL) and extracted with EtOAc (50.0 mL*2). The pH of the aqueous phase was adjusted to pH ~8 with sat. NaHCO₃ and extracted with CH₂Cl₂ (100 mL*3). The combined organic layers were washed with H₂O (50.0 mL*1), dried over Na₂SO₄, filtered and concentrated under reduced pressure. Compound Mavorixafor (1.10 g, 3.05 mmol, 45.8% yield, 97.0% purity) was obtained as a yellow solid, and confirmed by 1H NMR, LC-MS, HPLC, SFC, and OR. LC-MS (M+H)⁺: 350.2; ¹H NMR (400 MHZ, CDCl₃): δ 8.58 (d, J=4.0 Hz, 1H), 7.50-7.58 (m, 2H), 7.41 (d, J=7.2 Hz, 1H), 7.21-7.12 (m, 3H), 4.12-3.99 (m, 3H), 2.89-2.81 (m, 1H), 2.74-2.69 (m, 2H), 2.80-2.50 (m, 3H), 2.22-2.19 (m, 1H), 2.08-2.00 (m, 1H), 1.96-1.90 (m, 1H), 1.71-1.67 (m, 1H), 1.48-1.33 (m, 4H); HPLC purity: 97.0% (220 nm); SFC chiral purity: 100%.

### Example 4: Chiral Resolution of Mavorixafor Intermediates I

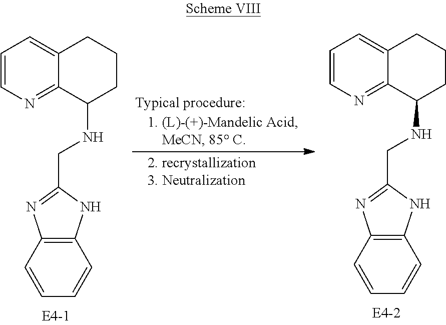 [SMILES: c1cnc2c(c1)CCCC2NCc1nc2ccccc2[nH]1 ; c1cnc2c(c1)CCC[C@H]2NCc1nc2ccccc2[nH]1]

TABLE I

| | | | | | | Times | | | |
|---|---|---|---|---|---|---|---|---|---|
| | | Chiral | Reaction | | Recrystallization | for | Weight of | Chiral | Isolated |
| | E4-1 | Acid | Solvent | Reaction | Solvent | Recrystallization | Product | Purity | Yield |
| Entry | (g) | (eq) | (mL/g) | Temp. | (mL/g) | (mL/g) | (g) | (214) | (%) |
| 1 | 3.5 | 2,3-Di- | DCM | 20-25 | iPrOH/THF | 2 | 2.3 g as salt | 13% | 27% |
| | | O-para- | (20) | | | | | ee | |
| | | toluoyl- | | | | | | | |
| | | D- | | | | | | | |
| | | tartaric | | | | | | | |
| | | acid/1.0 | | | | | | | |
| | | eq. | | | | | | | |
| 2 | 1.6 | N- | MeCN | 80 | MeCN | 2 | 1.0 g as salt | 3% | 38% |
| | | acetyl- | | | | | | ee | |
| | | L- | | | | | | | |
| | | leucine/ | | | | | | | |
| | | 1.0 eq. | | | | | | | |
| 3# | 0.5 | R-(−)- | MeCN | 85 | MeCN | 2 | 0.2 g as salt | ~100% | 21% |
| | | Camph | (20) | | | | | ee | |
| | | or-10- | | | | | | | |
| | | sulfonic | | | | | | | |
| | | Acid/1.0 | | | | | | | |
| | | eq. | | | | | | | |
| 4 | 0.5 | (S)-(+)- | MeCN | 85 | NA | NA | The salt is semi-solid, no | | |
| | | BNP/1.0 | (10) | | | | recrystallization exploration is | | |
| | | eq. | | | | | performed | | |
| 5 | 25.7 | R-(−)- | MeCN | 85 | MeCN | 2 | 16 g as salt | 3% | 32% |
| | | Camph | (20) | | | | | ee | |
| | | or-10- | | | | | | | |
| | | sulfonic | | | | | | | |
| | | Acid/1.0 | | | | | | | |
| | | eq. | | | | | | | |
| 6 | 0.5 | L-(+)- | MeCN | 80-85 | MeCN | 2 | 0.18 g as salt | 91% | 23% |
| | | Mandelic | (10) | | | | | ee | |
| | | Acid/ | | | | | | | |
| | | 1.0 eq. | | | | | | | |
| 7 | 10 | L-(+)- | MeCN | 80-85 | MeCN | 3 | 1.4 g as free | ~100% | 14% |
| | | Mandelic | (10) | | | | base | ee | |
| | | Acid/ | | | | | | | |
| | | 1.0 eq. | | | | | | | |
| 8 | 0.5 | R-(−)- | MeCN | 85 | MeCN | 2 | 0.22 g as salt | 4% | 22% |
| | | Camphor- | (20) | | | | | ee | |
| | | 10- | | | | | | | |
| | | sulfonic | | | | | | | |
| | | Acid/1.0 | | | | | | | |
| | | eq. | | | | | | | |
| 9 | 80 | D-(−)- | MeCN | 85 | MeCN | 3 | 13.6 as free | 94.5% | 19.6% |
| | | Mandelic | (20) | | | | base | ee | |
| | | Acid/ | | | | | | | |
| | | 1.0 eq. | | | | | | | |

### Experimental Data for Entries:

Entry 5: Resolution with R-(−)-Camphor-10-Sulfonic Acid:

To a solution of N-((1H-benzo[d]imidazol-2-yl)methyl)-5,6,7,8-tetrahydroquinolin-8-amine (25.7 g, 92 mmol) in MeCN was added R-(−)-camphor-10-sulfonic Acid (21.46 g, 92 mmol) at 20-25° C., then the mixture was heated to 80-85° C. and stirred at 80-85° C. for 0.5 h. The mixture was cooled to 20-25° C. and stirred at 20-25° C. for 3 hr. The solid was collected by filtration. The wet solid was added into MeCN (260 mL) and then the mixture was heated to 80-85° C. and stirred at 80-85° C. for 0.5 h. The mixture was cool to 20-25° C. and stirred at 20-25° C. for 3 hr. The solid was collected by filtration and dried under high vacuum by oil pump to give the salt (16 g, 32%) with 3% ee.

Entry 6: Resolution with L-(+)-Mandelic Acid:

To a solution of N-((1H-benzo[d]imidazol-2-yl)methyl)-5,6,7,8-tetrahydroquinolin-8-amine (0.5 g, 1.8 mmol) was added L-(+)-Mandelic Acid (0.27 g, 1.8 mmol) at 20-25° C., and then the mixture was heated to 80-85° C. and stirred at 80-85° C. for 0.5 hr. The mixture was cooled to 20-25° C. and stirred at 20-25° C. for 3 h. The solid was collected by filtration. The wet solid was added into MeCN (6 mL) and then the mixture was heated to 80-85° C. and stirred at 80-85° C. for 0.5 h. The mixture was cooled to 20-25° C. and stirred at 20-25° C. for 3 hr. The solid was collected by filtration and dried under high vacuum by oil pump to give the salt (0.18 g, 23%) with 91% ee.

Entry 7: Resolution with L-(+)-Mandelic Acid:

To a solution of N-((1H-benzo[d]imidazol-2-yl)methyl)-5,6,7,8-tetrahydroquinolin-8-amine (10 g, 36 mmol) was added L-(+)-Mandelic Acid (5.47 g, 36 mmol) at 20-25° C., and then the mixture was heated to 80-85° C. and stirred at 80-85° C. for 0.5 h. The mixture was cool to 20-25° C. and stirred at 20-25° C. The solid was collected by filtration. The wet solid was added into MeCN (100 mL) and then the mixture was heated to 80-85° C. and stirred at 80-85° C. for 0.5 h. The mixture was cooled to 20-25° C. and stirred at 20-25° C. for 3 hr and the solid was collected by filtration. The wet solid was added into MeCN (60 mL) and then the mixture was heated to 80-85° C. and stirred at 80-85° C. for 0.5 h. The mixture was cooled to 20-25° C. and stirred at 20-25° C. for 3 hr and the solid was collected by filtration. The wet solid was suspended between DCM (100 mL) and water (60 mL) and neutralized by NaOH to pH 10-11. The organic phase was washed with brine (40 mL), dried over Na₂SO₄, filtered, and concentrated to give the free base (1.4 g, 14%) with ~100% ee.

Entry 8: Resolution with R-(−)-Camphor-10-Sulfonic Acid:

To a solution of N-((1H-benzo[d]imidazol-2-yl)methyl)-5,6,7,8-tetrahydroquinolin-8-amine (0.5 g, 1.8 mmol) was added R-(−)-Camphor-10-sulfonic Acid (0.42 g, 1.8 mmol) at 20-25° C., and then the mixture was heated to 80-85° C. and stir at 80-85° C. for 0.5 hr. The mixture was cooled to 20-25° C. and stirred at 20-25° C. The solid was collected by filtration. The wet solid was added into MeCN (5 mL) and then the mixture was heated to 80-85° C. and stirred at 80-85° C. for 0.5 hr. The mixture was cooled to 20-25° C. and stirred at 20-25° C. for 3 hr. The solid was collected by filtration and dried under high vacuum by oil pump to give the salt (0.22 g, 22%) with 3.98% ee.

### Example 5: Chiral Resolution of Mavorixafor Intermediates II

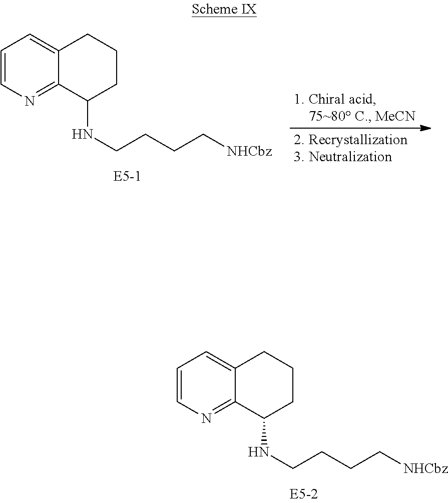 [SMILES: O=C(NCCCCNC1CCCc2cccnc21)OCc1ccccc1 ; O=C(NCCCCN[C@H]1CCCc2cccnc21)OCc1ccccc1]

^{&}: Compound E5-1 with 96% LCMS (clean 1HNMR) purity is used for screen.
^{#}: Compound E5-1 with 95% LCMS (~90% 1HNMR) purity is used for screen.

| | | | | | | Weight | | |
|---|---|---|---|---|---|---|---|---|
| | | | Reaction | Reaction | Recrystallization | of | Chiral | Isolated |
| | E5-1 | | Solvent | Temp. | Solvent | Product | Purity | Yield |
| Entry | (g) | Chiral Acid (eq) | (mL/g) | (° C.) | (mL/g) | (g) | -214 | (%) |
| 1^{#} | 0.5 | N-Boc-D-proline | MeCN(14) | 15-80 | No solid salt | | | |
| | | /1.0 eq. | | | | | | |
| 2^{#} | 0.5 | (S)-(+)-Mandelic | MeCN(14) | 15-80 | No solid salt | | | |
| | | acid/1.0 eq. | | | | | | |
| 3^{#} | 0.3 | L-Malic acid/1.0 eq. | MeCN(14) | 15-80 | No solid salt | | | |
| 4^{#} | 0.75 | N-Ac-L-Leu/1.0 eq. | MeCN(10) | 15-80 | Trace solid | | | |
| 5^{#} | 0.75 | N-Ac-L-Pro/1.0 eq. | MeCN(10) | 15-80 | Trace solid | | | |
| 6^{#} | 0.75 | L-tartaric acid/1.0 eq. | MeCN(10) | 15-80 | As oil | 0.7 g | ~0% | 65% |
| | | | | | | | ee | |
| 7^{#} | 0.75 | L-tartaric acid/1.0 eq. | iPrOH(17) | 15-80 | No solid salt | | | |
| 8^{#} | 0.75 | L-Malic acid/1.0 eq. | iPrOH(7) | 15-80 | No solid salt | | | |
| 9^{#} | 0.5 | (−)-Di-p-toluoyl-L- | MeCN(7) | 15-80 | No solid salt | | | |
| | | tartaric acid/1.0 eq. | | | | | | |
| 10# | 0.5 | N-Ts-L-Pro/1.0 eq. | MeCN(10) | 15-80 | No solid salt | | | |
| 11# | 0.75 | N-Ac-L-Pro/1.0 eq. | EtOH(10) | 15-80 | No solid salt | | | |
| 12# | 0.5 | N-Ts-L-Pro/1.0 eq. | iPrOH(10) | 15-80 | No solid salt | | | |
| 13# | 0.5 | L-tartaric acid/1.0 eq. | iPrOH/EA | 15-80 | No solid salt | | | |
| | | | (10/5) | | | | | |
| 14# | 0.5 | (−)-Di-p-toluoy1-L- | MeCN/EA | 15-80 | Trace solid | 0.11 g | ~0% | 10% |
| | | tartaric acid /1.0 eq. | (7/3.5) | | | | ee | |
| 15# | 0.5 | L-Malic acid/1.0 eq. | EtOH/EA | 15-80 | No solid salt | | | |
| | | | (10/5) | | | | | |
| 17# | 0.75 | N-Ac-L-Leu/1.0 eq. | iPrOH(10) | 15-80 | No solid salt | | | |
| 18# | 0.75 | N-Ac-L-Pro/1.0 eq. | EtOH/EA | 15-80 | No solid salt | | | |
| | | | (10/5) | | | | | |
| 19* | 0.05 | N-Boc-D-proline /1.0 eq. | MeCN(10) | 15-80 | No solid salt | | | |
| 20* | 0.05 | (S)-(+)-Mandelic acid/1.0 eq. | MeCN(10) | 15-80 | No solid salt | | | |
| 21* | 0.05 | L-Malic acid/1.0 eq. | MeCN(10) | 15-80 | As oil | | | |
| 22* | 0.05 | N-Ac-L-Leu/1.0 eq. | MeCN(10) | 15-80 | NA | ~50 mg | ~3% | NA |
| | | | | | | | ee | |
| 23* | 0.05 | N-Ac-L-Pro/1.0 eq. | MeCN(10) | 15-80 | No solid salt | | | |
| 24* | 0.05 | L-tartaric acid/1.0 eq. | MeCN(10) | 15-80 | As oil | | | |
| 25* | 0.05 | N-Ts-L-Pro/1.0 eq. | MeCN(10) | 15-80 | No solid salt | | | |
| 26* | 0.05 | (S)-BNP acid/1.0eq. | MeCN(10) | 15-80 | As oil | | | |
| 27* | 0.05 | (−)-Di-p-toluoyl-L- | MeCN(10) | 15-80 | No solid salt | | | |
| | | tartaric acid /1.0 eq. | | | | | | |
| 29* | 0.05 | (1S)-(+)-10- | MeCN(10) | 15-80 | No solid salt | | | |
| | | Camphorsulfonicacid/1.0 eq. | | | | | | |
| 30* | 0.05 | Di-p-anisoy1-D- | MeCN(10) | 15-80 | As oil | | | |
| | | tartaric acid/1.0 eq. | | | | | | |
| 31* | 0.05 | Dibenzoyl-L- | MeCN(10) | 15-80 | As oil | | | |
| | | tartaric acid/1.0eq. | | | | | | |
| 32* | 0.05 | N-Boc-L-alanine | MeCN(10) | 15-80 | No solid salt | | | |
| | | /1.0 eq. | | | | | | |
| 33* | 0.05 | L-Pyroglutamic | MeCN(10) | 15-80 | No solid salt | | | |
| | | acid/1.0 eq. | | | | | | |
| 34* | 0.05 | (1S)-(−)-Camphanic | MeCN(10) | 15-80 | No solid salt | | | |
| | | acid/1.0 eq. | | | | | | |
| 35* | 0.05 | N-Acetyl-L- | MeCN(10) | 15-80 | As oil | | | |
| | | tyrosine/1.0 eq. | | | | | | |
| 36* | 0.05 | (s)-Naproxen/1.0 eq. | MeCN(10) | 15-80 | No solid salt | | | |
| 36^{&} | 0.05 | N-Boc-D-Pro/1.0 eq. | Acetone(10) | 15-56 | No solid salt | | | |
| 37^{&} | 0.05 | (S)-(+)-Mandelic | Acetone(10) | 15-56 | No solid salt | | | |
| | | acid/1.0 eq. | | | | | | |
| 38^{&} | 0.05 | L-Malic acid/1.0 eq. | Acetone(10) | 15-56 | No solid salt | | | |
| 39^{&} | 0.05 | (s)-Naproxen/1.0 eq. | Acetone(10) | 15-56 | No solid salt | | | |
| 40^{&} | 0.05 | N-Ac-L-Leu/1.0 eq. | Acetone(10) | 15-56 | No solid salt | | | |
| 41^{&} | 0.05 | N-Ac-L-Pro/1.0 eq. | Acetone(10) | 15-56 | No solid salt | | | |
| 42^{&} | 0.05 | L-tartaric acid/1.0 eq. | Acetone(10) | 15-56 | No solid salt | | | |
| 43^{&} | 0.05 | N-Ts-L-Pro/1.0 eq. | Acetone(10) | 15-56 | No solid salt | | | |
| 44^{&} | 0.05 | (S)-BNP acid/1.0 eq. | Acetone(10) | 15-56 | No solid salt | | | |
| 45^{&} | 0.05 | (−)-Di-p-toluoy1-L- | Acetone(10) | 15-56 | No solid salt | | | |
| | | tartaric acid/1.0 eq. | | | | | | |
| 46^{&} | 0.05 | (1S)-(+)-10- | Acetone(10) | 15-56 | No solid salt | | | |
| | | Camphorsulfonicacid/1.0 eq. | | | | | | |
| 47^{&} | 0.05 | Di-p-anisoyl-D- | Acetone(10) | 15-56 | No solid salt | | | |
| | | tartaric acid/1.0 eq. | | | | | | |
| 48^{&} | 0.05 | Dibenzoyl-L- | Acetone(10) | 15-56 | No solid salt | | | |
| | | tartaric acid/1.0 eq. | | | | | | |
| 49^{&} | 0.05 | N-Boc-L- alanine/1.0 eq. | Acetone(10) | 15-56 | No solid salt | | | |
| 50^{&} | 0.05 | L-Pyroglutamic acid/1.0 eq. | Acetone(10) | 15-56 | No solid salt | | | |
| 51^{&} | 0.05 | (1S)-(−)-Camphanic | Acetone(10) | 15-56 | No solid salt | | | |
| | | acid/1.0 eq. | | | | | | |
| 52^{&} | 0.05 | N-Ac-L- tyrosine/1.0 eq. | Acetone(10) | 15-56 | No solid salt | | | |
| 53& | 0.05 | N-Boc-D-Pro/1.0 eq. | EtOH(10) | 15-80 | No solid salt | | | |
| 54& | 0.05 | (S)-(+)-Mandelic | EtOH(10) | 15-80 | No solid salt | | | |
| | | acid/1.0 eq. | | | | | | |
| 55& | 0.05 | L-Malic acid/1.0 eq. | EtOH(10) | 15-80 | No solid salt | | | |
| 56& | 0.05 | (s)-Naproxen/1.0 eq. | EtOH(10) | 15-80 | No solid salt | | | |
| 57& | 0.05 | N-Ac-L-Leu/1.0 eq. | EtOH(10) | 15-80 | No solid salt | | | |
| 58& | 0.05 | N-Ac-L-Pro/1.0 eq. | EtOH(10) | 15-80 | No solid salt | | | |
| 59& | 0.05 | L-tartaric acid/1.0 eq. | EtOH(10) | 15-80 | No solid salt | | | |
| 60& | 0.05 | N-Ts-L-Pro/1.0 eq. | EtOH(10) | 15-80 | No solid salt | | | |
| 61& | 0.05 | (S)-BNP acid/1.0 eq. | EtOH(10) | 15-80 | No solid salt | | | |
| 62& | 0.05 | (−)-Di-p-toluoy1-L- | EtOH(10) | 15-80 | No solid salt | | | |
| | | tartaric acid/1.0 eq. | | | | | | |
| 63& | 0.05 | (1S)-(+)-10- | EtOH(10) | 15-80 | No solid salt | | | |
| | | Camphorsulfonicacid/1.0 eq. | | | | | | |
| 64& | 0.05 | Di-p-anisoy1-D- | EtOH(10) | 15-80 | No solid salt | | | |
| | | tartaric acid/1.0 eq. | | | | | | |
| 65& | 0.05 | Dibenzoyl-L- | EtOH(10) | 15-80 | No solid salt | | | |
| | | tartaric acid/1.0 eq. | | | | | | |
| 66& | 0.05 | N-Boc-L- alanine/1.0 eq. | EtOH(10) | 15-80 | No solid salt | | | |
| 67& | 0.05 | L-Pyroglutamic acid/1.0 eq. | EtOH(10) | 15-80 | No solid salt | | | |
| 68& | 0.05 | (1S)-(−)-Camphanic | EtOH(10) | 15-80 | No solid salt | | | |
| | | acid/1.0 eq. | | | | | | |
| 69& | 0.05 | N-Ac-L- tyrosine/1.0 eq. | EtOH(10) | 15-80 | No solid salt | | | |
| 70& | 0.05 | N-Boc-D-Pro/1.0 eq. | iPrOH(10) | 15-80 | No solid salt | | | |
| 71& | 0.05 | (S)-(+)-Mandelic | iPrOH(10) | 15-80 | No solid salt | | | |
| | | acid/1.0 eq. | | | | | | |
| 72& | 0.05 | L-Malic acid/1.0 eq. | iPrOH(10) | 15-80 | No solid salt | | | |
| 73& | 0.05 | (s)-Naproxen/1.0 eq. | iPrOH(10) | 15-80 | No solid salt | | | |
| 74& | 0.05 | N-Ac-L-Leu/1.0 eq. | iPrOH(10) | 15-80 | No solid salt | | | |
| 75& | 0.05 | N-Ac-L-Pro/1.0 eq. | iPrOH(10) | 15-80 | No solid salt | | | |
| 76& | 0.05 | L-tartaric | iPrOH(10) | 15-80 | As soild | ~20 mg | ~0 | NA |
| | | acid/1.0 eq. | | | | | ee % | |
| 77& | 0.05 | N-Ts-L-Pro/1.0 eq. | iPrOH(10) | 15-80 | No solid salt | | | |
| 78& | 0.05 | (S)-BNP acid/1.0 eq. | iPrOH(10) | 15-80 | As oil | ~20 mg | ~0 | NA |
| | | | | | | | ee % | |
| 79& | 0.05 | (−)-Di-p-toluoy1-L- | iPrOH(10) | 15-80 | No solid salt | | | |
| | | tartaric acid/1.0 eq. | | | | | | |
| 80& | 0.05 | (1S)-(+)-10- | iPrOH(10) | 15-80 | No solid salt | | | |
| | | Camphorsulfonicacid/1.0 eq. | | | | | | |
| 81& | 0.05 | Di-p-anisoy1-D- | iPrOH(10) | 15-80 | As soild | ~20 mg | ~0 | NA |
| | | tartaric acid/1.0 eq. | | | | | ee % | |
| 82& | 0.05 | Dibenzoyl-L- | iPrOH(10) | 15-80 | As oil | ~20 mg | ~0 | NA |
| | | tartaric acid/1.0 eq. | | | | | ee % | |
| 83& | 0.05 | N-Boc-L- | iPrOH(10) | 15-80 | No solid salt | | | |
| | | alanine/1.0 eq. | | | | | | |
| 84& | 0.05 | L-Pyroglutamic | iPrOH(10) | 15-80 | No solid salt | | | |
| | | acid/1.0 eq. | | | | | | |
| 85& | 0.05 | (1S)-(−)-Camphanic | iPrOH(10) | 15-80 | No solid salt | | | |
| | | acid/1.0 eq. | | | | | | |
| 86& | 0.05 | N-Ac-L- | iPrOH(10) | 15-80 | No solid salt | | | |
| | | tyrosine/1.0 eq. | | | | | | |

### Example 6: Chiral Resolution of Mavorixafor Intermediates I

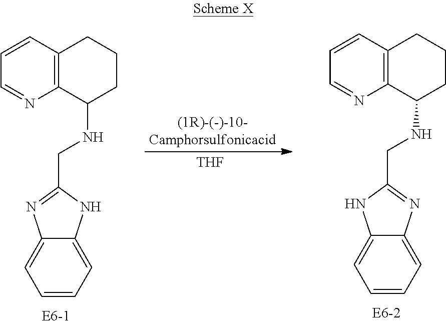 [SMILES: c1cnc2c(c1)CCCC2NCc1nc2ccccc2[nH]1 ; c1cnc2c(c1)CCC[C@@H]2NCc1nc2ccccc2[nH]1 ; "THF"]

| | | | | | | | Target/ | |
|---|---|---|---|---|---|---|---|---|
| | | | | | | Target/ | Isomer | |
| Material | Chiral | | | | | Isomer | (Monther | |
| (E6-1) | Reagent | CAS | Equivalent | Solvent | Result | (Precipitate) | liquid) | Yield |
| 50 mg | L-(+)- | 87- | 0.5 eq | EtOH | Oil | | | |
| | Tartaricacid | 69-4 | | (0.7 | | | | |
| | | | | mL) | | | | |
| 50 mg | (−)-Di-p- | 3263 | 0.5 eq | EtOH | Clear | | | |
| | toluoyl-L- | 4-66- | | (0.7 | | | | |
| | tartaric acid | 5 | | mL) | | | | |
| 50 mg | (−)-Dibenzoyl- | 2743- | 0.5 eq | EtOH | Clear | | | |
| | L-tartaric Acid | 38-6 | | (0.7 | | | | |
| | | | | mL) | | | | |
| 50 mg | (+)-Di-p- | 1916 | 0.5 eq | EtOH | Oil | | | |
| | anisoyl-D- | 05- | | (0.7 | | | | |
| | tartaric Acid | 10-4 | | mL) | | | | |
| 50 mg | (S)-(+)- | 1719 | 0.5 eq | EtOH | Clear | | | |
| | Mandelic Acid | 9-29- | | (0.7 | | | | |
| | | 0 | | mL) | | | | |
| 50 mg | (+)-Camphoric | 124- | 0.5 eq | EtOH | Clear | | | |
| | Acid | 83-4 | | (0.7 | | | | |
| | | | | mL) | | | | |
| 50 mg | L-Pyroglutamic | 98- | 0.5 eq | EtOH | Clear | | | |
| | Acid | 79-3 | | (0.7 | | | | |
| | | | | mL) | | | | |
| 50 mg | (S)-2-(6- | 2220 | 0.5 eq | EtOH | Clear | | | |
| | Methoxynaphthalen- | 4-53- | | (0.7 | | | | |
| | 2- | 1 | | mL) | | | | |
| | yl)propanoic | | | | | | | |
| | acid | | | | | | | |
| 50 mg | N-Boc-L- | 1576 | 0.5 eq | EtOH | Clear | | | |
| | alanine | 1-38- | | (0.7 | | | | |
| | | 3 | | mL) | | | | |
| 50 mg | N-Acetyl-L- | 537- | 0.5 eq | EtOH | Clear | | | |
| | tyrosine | 55-3 | | (0.7 | | | | |
| | | | | mL) | | | | |
| 50 mg | N-Acetyl-L- | 1188- | 0.5 eq | EtOH | Clear | | | |
| | leucine | 21-2 | | (0.7 | | | | |
| | | | | mL) | | | | |
| 50 mg | (1S)-(+)-10- | 3144- | 0.5 eq | EtOH | Suspension | | | |
| | Camphorsulfonicacid | 16-9 | | (0.7 | | | | |
| | | | | mL) | | | | |
| 50 mg | L-(+)- | 87- | 0.5 eq | THF | Trace | | | |
| | Tartaricacid | 69-4 | | (0.7 | solid | | | |
| | | | | mL) | | | | |
| 50 mg | (−)-Di-p- | 3263 | 0.5 eq | THF | Clear | | | |
| | toluoyl-L- | 4-66- | | (0.7 | | | | |
| | tartaric acid | 5 | | mL) | | | | |
| 50 mg | (−)-Dibenzoyl- | 2743- | 0.5 eq | THF | Clear | | | |
| | L-tartaric Acid | 38-6 | | (0.7 | | | | |
| | | | | mL) | | | | |
| 50 mg | (+)-Di-p- | 1916 | 0.5 eq | THF | Suspension | | | |
| | anisoyl-D- | 05- | | (0.7 | | | | |
| | tartaric Acid | 10-4 | | mL) | | | | |
| 50 mg | (S)-(+)- | 1719 | 0.5 eq | THF | Clear | | | |
| | Mandelic Acid | 9-29- | | (0.7 | | | | |
| | | 0 | | mL) | | | | |
| 50 mg | (+)-Camphoric | 124- | 0.5 eq | THF | Suspension | | | |
| | Acid | 83-4 | | (0.7 | | | | |
| | | | | mL) | | | | |
| 50 mg | L-Pyroglutamic | 98- | 0.5 eq | THF | Suspension | | | |
| | Acid | 79-3 | | (0.7 | | | | |
| | | | | mL) | | | | |
| 50 mg | (S)-2-(6- | 2220 | 0.5 eq | THF | Suspension | | | |
| | Methoxynaphthalen- | 4-53- | | (0.7 | | | | |
| | 2- | 1 | | mL) | | | | |
| | yl)propanoic | | | | | | | |
| | acid | | | | | | | |
| 50 mg | N-Boc-L- | 1576 | 0.5 eq | THF | Suspension | | | |
| | alanine | 1-38- | | (0.7 | | | | |
| | | 3 | | mL) | | | | |
| 50 mg | N-Acetyl-L- | 537- | 0.5 eq | THF | Suspension | | | |
| | tyrosine | 55-3 | | (0.7 | | | | |
| | | | | mL) | | | | |
| 50 mg | N-Acetyl-L- | 1188- | 0.5 eq | THF | Solid | 44.30/55.70 | / | / |
| | leucine | 21-2 | | (0.7 | | | | |
| | | | | mL) | | | | |
| 50 mg | (1S)-(+)-10- | 3144- | 0.5 eq | THF | Solid | 0/100 | 71.78/28.22 | 44.47% |
| | Camphorsulfonicacid | 16-9 | | (0.7 | | | | |
| | | | | mL) | | | | |
| 50 mg | (1R)-(−)-10- | 3596 | 0.5 eq | THF | Solid | 92.15/7.85 | 20.15/79.85 | 25.23% |
| | Camphorsulfonicacid | 3-20- | | (0.7 | | | | |
| | | 3 | | mL) | | | | |

We claim:

1. A method for preparing mavorixafor: 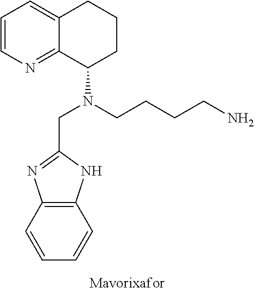 [SMILES: NCCCCN(Cc1nc2ccccc2[nH]1)[C@H]1CCCc2cccnc21]
or a pharmaceutically acceptable salt thereof;
comprising the steps of:
(a) coupling a compound of formula A: 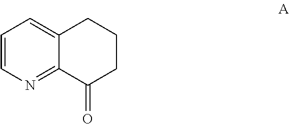 [SMILES: O=C1CCCc2cccnc21]
or a salt thereof;
with a compound of formula C: 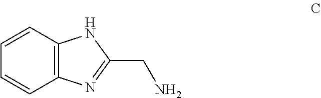 [SMILES: NCc1nc2ccccc2[nH]1]
or a salt thereof;
to prepare a compound of formula D: 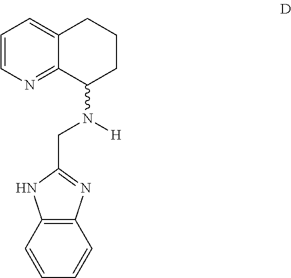 [SMILES: c1cnc2c(c1)CCCC2NCc1nc2ccccc2[nH]1]
or a salt thereof;
(b) purifying the compound of formula D to prepare the enantiomerically enriched compound of formula E: 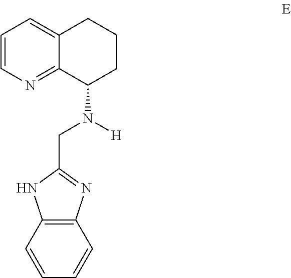 [SMILES: c1cnc2c(c1)CCC[C@@H]2NCc1nc2ccccc2[nH]1]
or a salt thereof;
(c) coupling the compound of formula E with a compound of formula F: 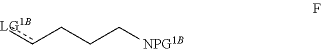
or a salt thereof;
wherein:
LG^{1B} is a suitable leaving group, ═O, or, taken with the carbon atom to which it is bound, an acetal; and
PG^{1B} is any combination of two amino protecting groups, any single amino protecting group and H, or two H; or PG^{1B} is, taken with the nitrogen to which it is bound, a cyclic imide; or PG^{1B} is absent and a triple bond is present between the corresponding nitrogen and the carbon to which the nitrogen is bound such that a nitrile is present;
to prepare a compound of formula G: 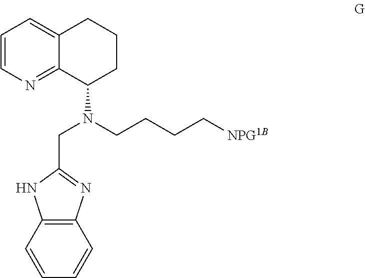
or salt thereof; and
(d) deprotecting the compound of formula G to form mavorixafor or a pharmaceutically acceptable salt thereof.

2. The method according to claim 1, wherein PG^{1B} is absent and a triple bond is present between the corresponding nitrogen and the carbon to which the nitrogen is bound, such that the nitrogen and the carbon to which it is bound form a nitrile.

3. The method according to claim 1, wherein PG^{1B} is Boc and H.

4. The method according to claim 1, wherein coupling of the compound of formula A and coupling of the compound of formula C to prepare the compound of formula D is achieved through reductive amination.

5. The method according to claim 1, wherein coupling of the compound of formula E and coupling of the compound of formula F to prepare the compound of formula G is achieved through reductive amination.

6. The method according to any one of claims 1-5, wherein purification of the compound of formula D to prepare the enantiomerically enriched compound of formula E is achieved through crystallization using a chiral acid in a suitable solvent.

7. The method according to any one of claims 1-5, wherein purification of the compound of formula D to prepare the enantiomerically enriched compound of formula E is achieved through crystallization using (R)-(−)-mandelic acid or (1R)-(−)-10-camphorsulofnic acid in a suitable solvent.

8. The method according to any one of claims 1-5, wherein purification of the compound of formula D to prepare the enantiomerically enriched compound of formula E is achieved by dissolving the compound of formula D in MeCN at a concentration of up 0.25 g/mL with up to 2.0 equivalents of (R)-(−)-mandelic acid added.

9. The method according to any one of claims 1-5, wherein purification of the compound of formula D to prepare the enantiomerically enriched compound of formula E is achieved by dissolving the compound of formula D in MeCN at a concentration of up 0.25 g/mL, to which up to 2.0 equivalents of (R)-(−)-mandelic acid is added at room temperature, at which point it is heated to an elevated temperature and held there for a period of time before it is cooled down to room temperature for a period of time.

10. The method according to any one of claims 1-5, wherein purification of the compound of formula D to prepare the enantiomerically enriched compound of formula E is achieved by dissolving the compound of formula D in MeCN at a concentration of up 0.25 g/mL, to which up to 2.0 equivalents of (R)-(−)-mandelic acid is added at a 15-25° C., at which point it is heated to a temperature between 65-85° C. and held at that temperature for 0.1 to 3 hour before it is cooled down to 0-25° C. and held at that temperature for 1 to 10 hours.

11. The method according to any one of claims 1-5, wherein purification of the compound of formula D to prepare the enantiomerically enriched compound of formula E is achieved by dissolving the compound of formula D in MeCN at a concentration of up 0.25 g/mL, to which up to 2.0 equivalents of (R)-(−)-mandelic acid is added at a 15-25° C., at which point it is heated to a temperature between 65-85° C. and held at that temperature for 0.1 to 3 hour before it is cooled down to 0-25° C. and held at that temperature for 1 to 10 hours, at which point the crystallized product is collected and used for additional rounds of recrystallizations using similar conditions, which yields the compound of formula E with an enantiomeric excess of at least 90%.

12. The method according to any one of claims 1-5, wherein purification of the compound of formula D to prepare the enantiomerically enriched compound of formula E is achieved by is dissolving the compound of formula D in MeCN at a concentration of up 0.25 g/mL, to which up to 2.0 equivalents of (R)-(−)-mandelic acid is added at 15-25° C., at which point it is heated to a temperature between 65-85° C. and held at that temperature for 0.1 to 3 hours before it is cooled down to 0-25° C. and held at that temperature for 1 to 10 hours, at which point the crystallized product is collected and used for additional rounds of recrystallizations using similar conditions, which yields the compound of formula E with an enantiomeric excess of at least 95%.

13. The method according to any one of claims 1-5, wherein purification of the compound of formula D to prepare the enantiomerically enriched compound of formula E is achieved by is dissolving the compound of formula D in MeCN at a concentration of up 0.25 g/mL, to which up to 2.0 equivalents of (R)-(−)-mandelic acid is added at 15-25° C., at which point it is heated to a temperature between 65-85° C. and held at that temperature for 0.1 to 3 hours before it is cooled down to 0-25° C. and held at that temperature for 1 to 10 hours, at which point the crystallized product is collected and used for additional rounds of recrystallizations using similar conditions, which yields the compound of formula E with an enantiomeric excess of at least 99%.

14. The method according to any one of claims 1-5, wherein purification of the compound of formula D to prepare the enantiomerically enriched compound of formula E is achieved by dissolving the compound of formula D in MeCN at a concentration of up 0.11 g/mL with 1.0 equivalents of (R)-(−)-mandelic acid added.

15. The method according to any one of claims 1-5, wherein purification of the compound of formula D to prepare the enantiomerically enriched compound of formula E is achieved by dissolving the compound of formula D in MeCN at a concentration of up 0.11 g/mL, to which 1.0 equivalents of (R)-(−)-mandelic acid is added at room temperature, at which point it is heated to an elevated temperature and held there for a period of time before it is cooled down to room temperature for a period of time.

16. The method according to any one of claims 1-5, wherein purification of the compound of formula D to prepare the enantiomerically enriched compound of formula E is achieved by dissolving the compound of formula D in MeCN at a concentration of up 0.11 g/mL, to which 1.0 equivalents of (R)-(−)-mandelic acid is added at a 20-25° C., at which point it is heated to a temperature between 80-85° C. and held at that temperature for 0.5 hours before it is cooled down to 20-25° C. and held at that temperature for 3 hours.

17. The method according to any one of claims 1-5, wherein purification of the compound of formula D to prepare the enantiomerically enriched compound of formula E is achieved by dissolving the compound of formula D in MeCN at a concentration of up 0.11 g/mL, to which 1.0 equivalents of (R)-(−)-mandelic acid is added at 20-25° C., at which point it is heated to a temperature between 80-85° C. and held at that temperature for 0.5 hours before it is cooled down to 20-25° C. and held at that temperature for 3 hours, at which point the crystallized product is collected and used for additional rounds of recrystallizations using similar conditions, which yields the compound of formula E with an enantiomeric excess of at least 90%.

18. The method according to any one of claims 1-5, wherein purification of the compound of formula D to prepare the enantiomerically enriched compound of formula E is achieved by dissolving the compound of formula D in MeCN at a concentration of up 0.11 g/mL, to which 1.0 equivalents of (R)-(−)-mandelic acid is added at 20-25° C., at which point it is heated to a temperature between 80-85° C. and held at that temperature for 0.5 hours before it is cooled down to 20-25° C. and held at that temperature for 3 hours, at which point the crystallized product is collected and used for additional rounds of recrystallizations using similar conditions, which yields the compound of formula E with an enantiomeric excess of at least 95%.

19. The method according to any one of claims 1-5, wherein purification of the compound of formula D to prepare the enantiomerically enriched compound of formula E is achieved by dissolving the compound of formula D in MeCN at a concentration of up 0.11 g/mL, to which 1.0 equivalents of (R)-(−)-mandelic acid is added at 20-25° C., at which point it is heated to a temperature between 80-85° C. and held at that temperature for 0.5 hours before it is cooled down to 20-25° C. and held at that temperature for 3 hours, at which point the crystallized product is collected and used for additional rounds of recrystallizations using similar conditions, which yields the compound of formula E with an enantiomeric excess of at least 99%.

20. The method according to any one of claims 1-5, wherein purification of the compound of formula D to prepare the enantiomerically enriched compound of formula E is achieved through recrystallization using (1R)-(−)-10-camphorsulofnic acid in a suitable solvent.

21. The method according to any one of claims 1-5, wherein purification of the compound of formula D to prepare the enantiomerically enriched compound of formula E is achieved by dissolving the compound of formula D in THF at a concentration of up 0.2 g/mL, to which up to 1.5 equivalents of (1R)-(−)-10-camphorsulfonic is added.

22. The method according to any one of claims 1-5, wherein purification of the compound of formula D to prepare the enantiomerically enriched compound of formula E is achieved by dissolving the compound of formula D in THF at a concentration of up to 0.2 g/mL, to which up to 1.5 equivalents of (1R)-(−)-10-camphorsulfonic acid is added at room temperature and held at that temperature for a period of time, at which point it is heated to an elevated temperature and held at that temperature for a period of time, following which it is cooled to room temperature and held at that temperature for a period of time.

23. The method according to any one of claims 1-5, wherein purification of the compound of formula D to prepare the enantiomerically enriched compound of formula E is achieved by dissolving the compound of formula D in THF at a concentration of up to 0.2 g/mL, to which up to 1.5 equivalents of (1R)-(−)-10-camphorsulfonic acid is added at a temperature between 10-30° C. and held at that temperature for up to 3 hours before it is heated up to a temperature between 50-75° C. and held at that temperature for up to 3 hours, following which the mixture is cooled to a temperature between 0-25° C. and held at that temperature for up to 6 hours, following which the compound of formula E is collected with an enantiomeric excess of at least 90%.

24. The method according to any one of claims 1-5, wherein purification of the compound of formula D to prepare the enantiomerically enriched compound of formula E is achieved by dissolving the compound of formula D in THF at a concentration of up to 0.2 g/mL, to which up to 1.5 equivalents of (1R)-(−)-10-camphorsulfonic acid is added at a temperature between 10-30° C. and held at that temperature for up to 3 hours before it is heated up to a temperature between 50-75° C. and held at that temperature for up to 3 hours, following which the mixture is cooled to a temperature between 0-25° C. and held at that temperature for up to 6 hours, following which the compound of formula E is collected with an enantiomeric excess of at least 95%.

25. The method according to any one of claims 1-5, wherein purification of the compound of formula D to prepare the enantiomerically enriched compound of formula E is achieved by dissolving the compound of formula D in THE at a concentration of up to 0.2 g/mL, to which up to 1.5 equivalents of (1R)-(−)-10-camphorsulfonic acid is added at a temperature between 10-30° C. and held at that temperature for up to 3 hours before it is heated up to a temperature between 50-75° C. and held at that temperature for up to 3 hours, following which the mixture is cooled to a temperature between 0-25° C. and held at that temperature for up to 6 hours, following which the compound of formula E is collected with an enantiomeric excess of at least 99%.

26. The method according to any one of claims 1-5, wherein purification of the compound of formula D to prepare the enantiomerically enriched compound of formula E is achieved by dissolving the compound of formula D in THE at a concentration of up 0.1 g/mL, to which to 0.5 equivalents of (1R)-(−)-10-camphorsulfonic is added.

27. The method according to any one of claims 1-5, wherein purification of the compound of formula D to prepare the enantiomerically enriched compound of formula E is achieved by dissolving the compound of formula D in THF at a concentration of up to 0.1 g/mL, to which 0.5 equivalents of (1R)-(−)-10-camphorsulfonic acid is added at room temperature and held at that temperature for a period of time, at which point it is heated to an elevated temperature and held at that temperature for a period of time, following which it is cooled and held at that temperature for a period of time.

28. The method according to any one of claims 1-5, wherein purification of the compound of formula D to prepare the enantiomerically enriched compound of formula E is achieved by dissolving the compound of formula D in THF at a concentration of up to 0.1 g/mL, to which 0.5 equivalents of (1R)-(−)-10-camphorsulfonic acid is added at 25° C. and held at that temperature for 1 hour before it is heated 70° C. and held at that temperature for 1 hour, following which the mixture is cooled to 25° C. and held at that temperature for 1 hour, following which the compound of formula E is collected with an enantiomeric excess of at least 90%.

29. The method according to any one of claims 1-5, wherein purification of the compound of formula D to prepare the enantiomerically enriched compound of formula E is achieved by dissolving the compound of formula D in THF at a concentration of up to 0.1 g/mL, to which 0.5 equivalents of (1R)-(−)-10-camphorsulfonic acid is added at 25° C. and held at that temperature for 1 hour before it is heated 70° C. and held at that temperature for 1 hour, following which the mixture is cooled to 25° C. and held at that temperature for 1 hour, following which the compound of formula E is collected with an enantiomeric excess of at least 95%.

30. The method according to any one of claims 1-5, wherein purification of the compound of formula D to prepare the enantiomerically enriched compound of formula E is achieved by dissolving the compound of formula D in THF at a concentration of up to 0.1 g/mL, to which 0.5 equivalents of (1R)-(−)-10-camphorsulfonic acid is added at 25° C. and held at that temperature for 1 hour before it is heated 70° C. and held at that temperature for 1 hour, following which the mixture is cooled to 25° C. and held at that temperature for 1 hour, following which the compound of formula E is collected with an enantiomeric excess of at least 99%.

31. A method for preparing mavorixafor: 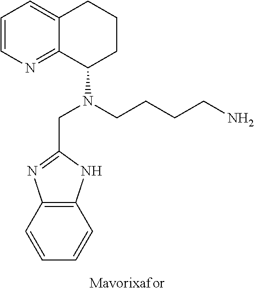 [SMILES: NCCCCN(Cc1nc2ccccc2[nH]1)[C@H]1CCCc2cccnc21]
or a pharmaceutically acceptable salt thereof;
comprising the steps of:
(a) coupling a compound of formula A: 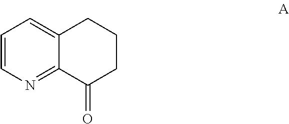 [SMILES: O=C1CCCc2cccnc21]
or a salt thereof;
with a compound of formula F′: 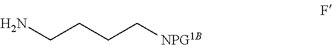
or a salt thereof;
wherein:
PG^{1B} is any combination of two amino protecting groups, any single amino protecting group and H, or two H; or PG^{1B} is, taken with the nitrogen to which it is bound, a cyclic imide; or PG^{1B} is absent and a triple bond is present between the corresponding nitrogen and the carbon to which the nitrogen is bound such that a nitrile is present;
to prepare a compound of formula D′: 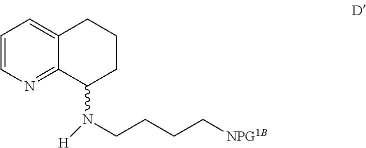
or salt thereof;
(b) purifying the compound of formula D′ to prepare the enantiomerically enriched compound of formula E′: 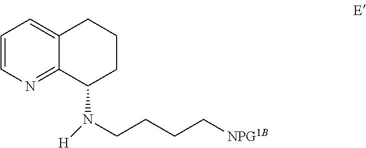
or salt thereof;
(c) coupling the compound of formula E′ with a compound of formula C′: 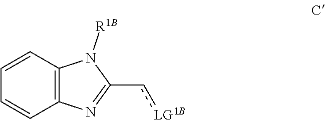
or salt thereof;
wherein:
R^{1B} is H, or a suitable benzimidazole protecting group; and
LG^{1B} is-O, a suitable leaving group, or, taken with the carbon atom to which it is bound, an acetal;
to prepare a compound of formula G: 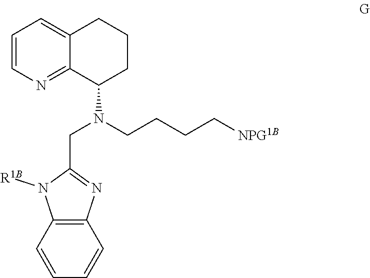
or salt thereof; and
(d) deprotecting the compound of formula G to form mavorixafor or a pharmaceutically acceptable salt thereof.

32. The method according to claim 31, wherein LG^{1B} is-O; R^{1B} is CH₂OCH₂Ph; and PG^{1B} is Boc and H.

33. The method according to claim 31, wherein coupling of the compound of formula A and coupling of the compound of formula F′ to prepare the compound of formula D′ is achieved through reductive amination.

34. The method according to claim 31, wherein coupling of the compound of formula E′ and coupling of the compound of formula C′ to prepare the compound of formula G is achieved through reductive amination.

35. The method according to any one of claims 32-34, wherein purification of the compound of formula D′ to prepare the enantiomerically enriched compound of formula E′ is achieved through crystallization using a chiral acid and a suitable solvent.

36. The method according to any one of claims 32-34, wherein purification of the compound of formula D′ to prepare the enantiomerically enriched compound of formula E′ is achieved through crystallization using a chiral amino acid and a suitable solvent.

37. The method according to any one of claims 32-34, wherein purification of the compound of formula D′ to prepare the enantiomerically enriched compound of formula E′ is achieved through recrystallization using N-Boc-D-proline, and MeCN.

38. The method according to any one of claims 32-34, wherein purification of the compound of formula D′ to prepare the enantiomerically enriched compound of formula E′ is achieved by dissolving the compound of formula D′ in MeCN at a concentration of up 0.15 g/mL, to which up to 1.5 equivalents of N-Boc-D-proline is added.

39. The method according to any one of claims 32-34, wherein purification of the compound of formula D′ to prepare the enantiomerically enriched compound of formula E′ is achieved by dissolving the compound of formula D′ in MeCN at a concentration of up to 0.15 g/mL, to which up to 1.5 equivalents of N-Boc-D-proline is added at room temperature and held at that temperature for a period of time, at which point it is heated to an elevated temperature and held at that temperature for a period of time, following which it is cooled to room temperature and held at that temperature for a period of time.

40. The method according to any one of claims 32-34, wherein purification of the compound of formula D′ to prepare the enantiomerically enriched compound of formula E′ is achieved by dissolving the compound of formula D′ in MeCN at a concentration of up to 0.15 g/mL, to which up to 1.5 equivalents of N-Boc-D-proline is added at a temperature between 10-25° C. and held at that temperature for up to 3.0 hour before it is heated to 60-82° C. and held at that temperature for up to 3.0 hours, following which the mixture is cooled to a temperature between 0-25° C. and held at that temperature for up to 20 hours, following which the compound of formula E′ is collected with an enantiomeric excess of at least 90%.

41. The method according to any one of claims 32-34, wherein purification of the compound of formula D′ to prepare the enantiomerically enriched compound of formula E′ is achieved by dissolving the compound of formula D′ in MeCN at a concentration of up to 0.15 g/mL, to which up to 1.5 equivalents of N-Boc-D-proline is added at a temperature between 10-25° C. and held at that temperature for up to 3.0 hour before it is heated to 60-82° C. and held at that temperature for up to 3.0 hours, following which the mixture is cooled to a temperature between 0-25° C. and held at that temperature for up to 20 hours, following which the compound of formula E′ is collected with an enantiomeric excess of at least 95%.

42. The method according to any one of claims 32-34, wherein purification of the compound of formula D′ to prepare the enantiomerically enriched compound of formula E′ is achieved by dissolving the compound of formula D′ in MeCN at a concentration of up to 0.15 g/mL, to which up to 1.5 equivalents of N-Boc-D-proline is added at a temperature between 10-25° C. and held at that temperature for up to 3.0 hour before it is heated to 60-82° C. and held at that temperature for up to 3.0 hours, following which the mixture is cooled to a temperature between 0-25° C. and held at that temperature for up to 20 hours, following which the compound of formula E′ is collected with an enantiomeric excess of at least 99%.

43. The method according to any one of claims 32-34, wherein purification of the compound of formula D′ to prepare the enantiomerically enriched compound of formula E′ is achieved by dissolving the compound of formula D′ in MeCN at a concentration of up 0.07 g/mL, to which 0.5 equivalents of N-Boc-D-proline is added.

44. The method according to any one of claims 32-34, wherein purification of the compound of formula D′ to prepare the enantiomerically enriched compound of formula E′ is achieved by dissolving the compound of formula D′ in MeCN at a concentration of up to 0.07 g/mL, to which 0.5 equivalents of N-Boc-D-proline is added at room temperature and held at that temperature for a period of time, at which point it is heated to an elevated temperature and held at that temperature for a period of time, following which it is cooled to room temperature and held at that temperature for a period of time.

45. The method according to any one of claims 32-34, wherein purification of the compound of formula D′ to prepare the enantiomerically enriched compound of formula E′ is achieved by dissolving the compound of formula D′ in MeCN at a concentration of up to 0.07 g/mL, to which 0.5 equivalents of N-Boc-D-proline is added at a temperature between 20-25° C. and held at that temperature for 0.5 hours before it is heated to 75-80° C. and held at that temperature for 0.5 hours, following which the mixture is cooled to a temperature between 20-25° C. and held at that temperature for 16 hours, following which the compound of formula E′ is collected with an enantiomeric excess of at least 90%.

46. The method according to any one of claims 32-34, wherein purification of the compound of formula D′ to prepare the enantiomerically enriched compound of formula E′ is achieved by dissolving the compound of formula D′ in MeCN at a concentration of up to 0.07 g/mL, to which 0.5 equivalents of N-Boc-D-proline is added at a temperature between 20-25° C. and held at that temperature for 0.5 hours before it is heated to 75-80° C. and held at that temperature for 0.5 hours, following which the mixture is cooled to a temperature between 20-25° C. and held at that temperature for 16 hours, following which the compound of formula E′ is collected with an enantiomeric excess of at least 95%.

47. The method according to any one of claims 32-34, wherein purification of the compound of formula D′ to prepare the enantiomerically enriched compound of formula E′ is achieved by dissolving the compound of formula D′ in MeCN at a concentration of up to 0.07 g/mL, to which 0.5 equivalents of N-Boc-D-proline is added at a temperature between 20-25° C. and held at that temperature for 0.5 hours before it is heated to 75-80° C. and held at that temperature for 0.5 hours, following which the mixture is cooled to a temperature between 20-25° C. and held at that temperature for 16 hours, following which the compound of formula E′ is collected with an enantiomeric excess of at least 99%.

48. A method for preparing mavorixafor: 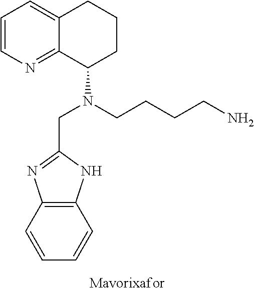 [SMILES: NCCCCN(Cc1nc2ccccc2[nH]1)[C@H]1CCCc2cccnc21]
or a pharmaceutically acceptable salt thereof;
comprising the steps of:
(a) coupling a compound of formula A: 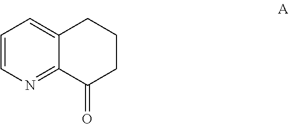 [SMILES: O=C1CCCc2cccnc21]
or a salt thereof;
with a compound of formula H: 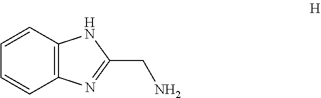 [SMILES: NCc1nc2ccccc2[nH]1]
or a salt thereof;
to prepare a compound of formula J: 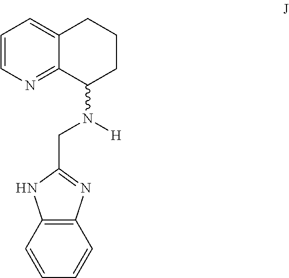 [SMILES: c1cnc2c(c1)CCCC2NCc1nc2ccccc2[nH]1]
or a salt thereof;
(b) coupling the compound of formula J with a compound of formula K: 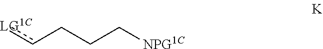
or a salt thereof;
wherein:
LG^{1C} is a suitable leaving group, ═O, or, taken with the carbon atom to which it is bound, an acetal; and
PG^{1C} is any combination of two amino protecting groups, any single amino protecting group and H, or two H; or PG^{1C} is, taken with the nitrogen to which it is bound, a cyclic imide; or PG^{1C} is absent and a triple bond is present between the corresponding nitrogen and the carbon to which the nitrogen is bound such that a nitrile is present;
to prepare a compound of formula L: 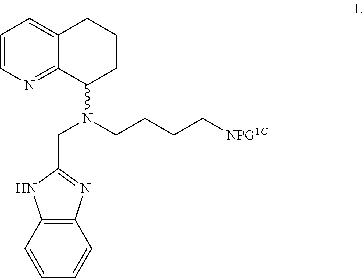
or salt thereof;
(c) purifying the compound of formula L to prepare the enantiomerically enriched compound of formula M: 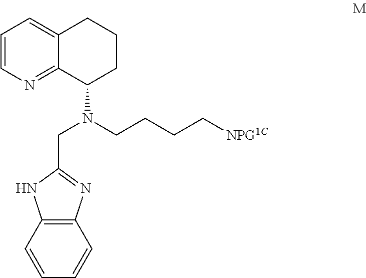
or salt thereof; and
(d) deprotecting the compound of formula M to form mavorixafor or a pharmaceutically acceptable salt thereof.

49. A method for preparing mavorixafor: 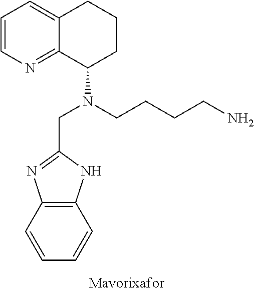 [SMILES: NCCCCN(Cc1nc2ccccc2[nH]1)[C@H]1CCCc2cccnc21]
or a pharmaceutically acceptable salt thereof;
comprising the steps of:
(a) coupling a compound of formula A: 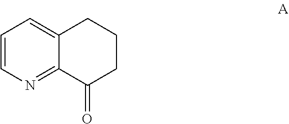 [SMILES: O=C1CCCc2cccnc21]
or a salt thereof;
with a compound of formula K′: 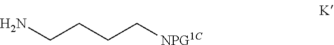
or salt thereof;
wherein:
PG^{1C} is any combination of two amino protecting groups, any single amino protecting group and H, or two H; or PG^{1C} is, taken with the nitrogen to which it is bound, a cyclic imide; or PG^{1C} is absent and a triple bond is present between the corresponding nitrogen and the carbon to which the nitrogen is bound such that a nitrile is present;
to prepare a compound of formula J′: 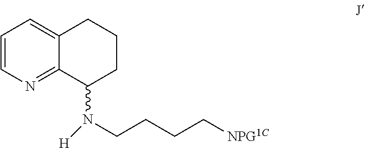
or salt thereof;
(b) coupling the compound of formula J′ with a compound of formula H′: 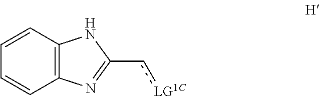
or salt thereof;
wherein:
LG^{1C} is ═O, a suitable leaving group, or, taken with the carbon atom to which it is bound, an acetal;
to prepare a compound of formula L: 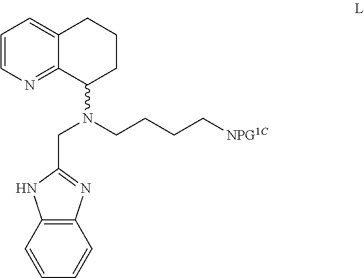
or salt thereof;
(c) purifying the compound of formula L to prepare the enantiomerically enriched compound of formula M: 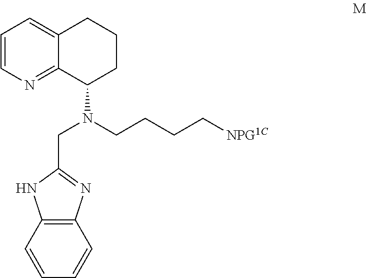
or salt thereof; and
(d) deprotecting the compound of formula M to form mavorixafor or a pharmaceutically acceptable salt thereof.

50. The method according to claim 48, wherein coupling the compound of formula A with the compound of formula H to prepare the compound of formula J is achieved through reductive amination.

51. The method according to claim 48, wherein coupling the compound of formula J with the compound of formula K to prepare the compound of formula L is achieved through reductive amination.

52. The method according to claim 49, wherein coupling the compound of formula A with the compound of formula K′ to prepare the compound of formula J′ is achieved through reductive amination.

53. The method according to claim 49, wherein coupling the compound of formula J′ with the compound of formula H′ to prepare the compound of formula L is achieved through reductive amination.

54. The method according to claim 48, wherein LG^{1C} is ═O; and PG^{1C} is H and Boc.

55. The method according to claim 49, wherein LG^{1C} is ═O; and PG^{1C} is H and Boc.

56. The method according to any one of claims 50-55, wherein purification of the compound of formula L to prepare the enantiomerically enriched compound of formula M is achieved by chiral HPLC.

57. The method according to any one of claims 50-55, wherein purification of the compound of formula L to prepare the enantiomerically enriched compound of formula M is achieved by chiral HPLC using a supercritical mobile phase.

58. The method according to any one of claims 50-55, wherein purification of the compound of formula L to prepare the enantiomerically enriched compound of formula M is achieved by chiral HPLC using a supercritical mobile phase composed of [CO₂-EtOH (0.1% NH₃H₂O)].

59. The method according to any one of claims 50-55, wherein purification of the compound of formula L to prepare the enantiomerically enriched compound of formula M is achieved by chiral HPLC using a supercritical mobile phase composed of [CO₂-EtOH (0.1% NH₃H₂O)] and the compound M is isolated with at least 90% enantiomeric excess.

60. The method according to any one of claims 50-55, wherein purification of the compound of formula L to prepare the enantiomerically enriched compound of formula M is achieved by chiral HPLC using a supercritical mobile phase composed of [CO₂-EtOH (0.1% NH₃H₂O)] and the compound M is isolated with at least 95% enantiomeric excess.

61. The method according to any one of claims 50-55, wherein purification of the compound of formula L to prepare the enantiomerically enriched compound of formula M is achieved by chiral HPLC using a supercritical mobile phase composed of [CO₂-EtOH (0.1% NH₃H₂O)] and the compound M is isolated with at least 99% enantiomeric excess.

62. A method for the purification of a compound of formula N: 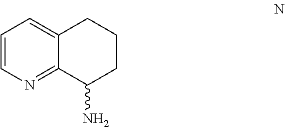 [SMILES: NC1CCCc2cccnc21]
or a salt thereof;
to prepare an enantiomerically enriched compound of formula O: 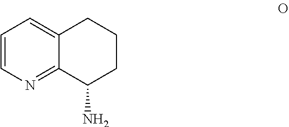 [SMILES: N[C@H]1CCCc2cccnc21]
or a salt thereof.

63. The method according claim 62, wherein the compound of formula N is purified to prepare the enantiomerically enriched compound of formula O by chiral HPLC.

64. The method according claim 62, wherein the compound of formula N is purified to prepare the enantiomerically enriched compound of formula O by crystallization with a chiral acid.

65. The method according either claim 63 or 64, wherein the compound of formula O is purified with an enantiomeric excess of at least 90%.

66. The method according either claim 63 or 64, wherein the compound of formula O is purified with an enantiomeric excess of at least 95%.

67. The method according either claim 63 or 64, wherein the compound of formula O is purified with an enantiomeric excess of at least 99%.
